(19) United States
(12) Patent Application Publication
(10) Pub. No.: US 2004/0091933 A1
(43) Pub. Date: May 13, 2004

(54) Methods for genetic interpretation and prediction of phenotype

(76) Inventors: Roland Stoughton, Kirkland, WA (US); Mattew J. Marton, Seattle, WA (US)

Correspondence Address: JONES DAY, 222 EAST 41ST STREET, NEW YORK, NY, 10017, US

(21) Appl. No.: 10/332,352

(22) PCT Filed: Jul. 2, 2001
(86) PCT No.: PCT/US01/20931

(51) Int. Cl. G01N 33/53

(52) U.S. Cl. 435/7.1

(57) ABSTRACT

The present invention relates to methods for determining the genetic causes of certain phenotypes. The present invention further relates to methods for predicting the phenotype of a organism from its genotype. In particular, the methods of the invention relate to the use of compendia of biological response profiles of cells having known genetic mutations for comparisons with the biological response profiles of cells having unknown phenotypes and genotypes. The methods of the present invention are particularly useful for monitoring the success of genetic engineering and cross-breeding of crops and livestock. The present invention further relates to a computer system for comparing biological response profiles to a compendium of biological response profiles and to kits for relating the phenotype of a cell type to its genotype or for predicting the phenotype of a cell type.

This application claims benefit of provisional U.S. Patent Application Serial No. 60/215,935 filed Jul. 5, 2000, which is incorporated by reference herein in its entirety.

### 1. FIELD OF THE INVENTION

The present invention relates to methods for determining which genes are responsible for certain phenotypes of interest. In particular, the present invention relates to the use of response profile libraries for monitoring the success of genetic engineering and cross-breeding attempts of crops and livestock.

### 2. BACKGROUND OF THE INVENTION

Genetic engineering of plants and livestock has led to advances in the production of agriculturally desirable phenotypes (Watson et al. (1992) Recombinant DNA 2^{nd} ed, W. H. Freeman and Co., New York). For example, plants have been developed that are resistant to disease, insects, and herbicides. Ornamental crops have been engineered to produce flowers that are bigger and more brightly-colored, or that have new colors, patterns and shapes. Plants may also be engineered to be drought resistant or frost resistant. Livestock animals have been engineered to be larger and leaner, and to use feed more efficiently. In addition, sheep may be engineered to produce more wool, and both livestock and companion animals may be engineered to be more disease resistant.

Genetic engineering of organisms requires knowing which genes are responsible for certain desirable phenotypes. Selective breeding by conventional cross-breeding techniques also benefits from this knowledge. However, current knowledge of the relationship of genotype to phenotype in most organisms is incomplete, and therefore, the usefulness of genetic engineering to produce a desired phenotype is limited.

The chromosomal locations of some genes have been determined by DNA sequencing of genomic DNA. For species whose genome has not been sequenced, genes can be approximately mapped as a result of co-inheritance linkage analysis (Sherman, F. and Wakem, P. (1991) Methods in Enzymology 194:38-57). For example, genes can be mapped by determining in what percentage of individuals they are co-inherited with a particular marker, such as a restriction fragment length polymorphism (“RFLP”) or a variable-number tandem repeat (“VNTR”) locus. These mapping procedures require large sets of multi-generational families with known phenotypes, and are much more difficult to perform successfully in the case of multi-genic traits (Tanksley, S. D. (1993), Mapping polygenes. Annu. Rev. Genet. 27, 205-233; Melchinger, A. E., Utz, H. F. and C. C. Schön. (1998), Quantitative trait locus (QTL) mapping using different testers and independent population samples in maize reveals low power of QTL detection and large bias in estimates of QTL effects, Genetics 149, 383-403; Paterson, A. H. (1995) Molecular dissection of quantitative traits: progress and prospects, Genome Research 5, 321-333; McCouch, S. R. and R. W. Doerge. (1995), QTL mapping in rice, Trends in Genetics 11, 482-487; Stuber, C. W. (1995), Mapping and manipulating quantitative traits in maize, Trends in Genetics 11, 477-481; Dupuis, J. and D. Siegmund. (1999), Statistical methods for mapping quantitative trait loci from a dense set of markers, Genetics 151, 373-386; Lander, E. S. and D. Botstein. (1989), Mapping Mendelian factors underlying quantitative traits using RFLP linkage maps, Genetics 121, 185-199, with erratum appearing in Genetics 136, 705 (1994)). Furthermore, once the closest marker to the gene of interest is identified, sequencing of the region from the marker to and including the gene is done by chromosome walking, whereby short stretches of overlapping sequences are elucidated and assembled to generate a longer sequence. If the marker is many bases away from the gene, the process of chromosome walking from marker to gene may take years. Finally, once the gene sequence is known, it must be proven through studies of many individuals that mutations in the gene of interest lead to the mutant phenotype.

A number of genotype-phenotype relationships have been determined in yeast and in plants by creating random genetic disruptions, observing the phenotype, and then screening for which gene was disrupted [Snyder, M., Elledge, S., and R W Davis. (1996), Rapid mapping of antigenic coding regions and constructing insertion mutations in yeast genes by mini-Tn10 “transplason” mutagenesis, Proc Natl Acad Sci USA 83,7304; Huisman, O., Raymond W., Froehlich K. U., Errada P., Keckner, N., Botstein, D., and M. A. Hoyt (1987), A Tn10-lacZ-URA3 gene fusion transposon for insertion mutagenesis and fusion analysis of yeast and bacterial genes, Genetics 116, 191-199; Garfinkel, D. J., Mastrangelo, M. F., Sanders, N. J., Shafer, B. K, and J. N. Strathem (1988), Transposon tagging using Ty elements in yeast, Genetics 120, 95-108; Erdman, S., Lui, L., Malczynski, M. and M Snyder (1998), Pheromone-regulated genes required for yeast mating differentiation, J Cell Biol 140, 461-483; Scott, K. F., Hughes, J. E., Gresshoff, P. M., Beringer, J. E., Rolfe, B. G., Shine (1982), Molecular cloning of Rhizobium trifolii genes involved in symbiotic nitrogen fixation, J Mol Appl Genet 1982; 1(4):315-26)]. The method of disrupting genes by making random insertions into the genome of an organism is now common. In contrast, it is difficult to make targeted, or known, genetic deletions, particularly in organisms with large or polyploid genomes. The number of genotype-phenotype relationships determined using random mutagenesis is usually very limited compared to the number of possible phenotypes. Some phenotypes generated from random mutagenesis are difficult to identify, and it may not be possible to obtain mutants in a particular desirable phenotype because mutations in the responsible gene are lethal events. Furthermore, the methods of locating randomly-inserted mutations require some effort.

Current methods of relating phenotype to genotype are cumbersome. For example, it is difficult to make mutations in organisms with large genomes or in organisms that are polyploid because all copies of a gene might have to be knocked out before a phenotype is observed. “reverse genetics” has been successful in laboratory yeast because the organism can survive as a haploid, which makes observing the effect of a mutation easier, as well as because the genome is small, rendering mutations easy to make. However, even in organisms such as Candida albicans, which are closely related to laboratory yeast, reverse genetics is difficult to perform because it is difficult to make knock-outs and the haploid organism does not grow. In addition, methods for relating phenotype to genotype either require large longitudinal studies in order to do the linkage analysis, or rely on detailed investigation of random mutations, the genomic locations of which must be identified, and their link to a phenotype proven. Furthermore, the methods become increasingly difficult as the complexity of the genome of the organism of interest increases. There is clearly a need for faster methods of relating phenotype to genotype that do not become proportionately difficult as the genome gets larger.

The methods of the present invention use expression profiles, which are measurements of cellular constituents, e.g., mRNA or protein species abundances, protein activities, levels of modification to protein such as phosphorylation of kinases, etc., as a phenotypic marker of a particular genotype before the actual mutations in those strains have been mapped. The transcript or protein abundance profile associated with a phenotype is compared with a library of landmark profiles, or “compendium”, obtained from known genetic perturbations in order to infer genetic cause. As a result, the effort required to map the mutations to specific genes can focus on strains having the phenotypes of interest. The methods of the present invention will facilitate genetic interpretation of genetic engineering or selective cross-breeding outcomes, the detection of unexpected genetic features in cross-breeding products, and more rapid identification of multiple genes contributing to a given trait.

Discussion or citation of a reference herein shall not be construed as an admission that such reference is prior art to the present invention.

### 3. SUMMARY OF THE INVENTION

The present invention provides methods for determining the genotype responsible for a particular phenotype, for relating a phenotype to a genotype of a cell type or organism, and for determining if a genotype associated with a particular phenotype is present in a cell type or organism. In particular, the present invention provides methods for relating genotype and phenotype by comparing an expression profile of a cell type or organism with a compendium of expression profiles of cell types or organisms having known genotypes and phenotypes. The present invention further relates to computer systems and computer program products for comparing an expression profile of a cell type or organism with a compendium of expression profiles of cell types or organisms having known genotypes and phenotypes.

In a first embodiment, the present invention relates to a method for determining one or more candidate genes, or their encoded RNAs or proteins, responsible for a phenotype of interest displayed by a cell type or organism, comprising: (a) determining measured amounts of a plurality of cellular constituents in a first cell of said cell type or of said organism to create a first profile; (b) comparing said first profile, or a predicted profile derived therefrom, to a database comprising a plurality of landmark profiles to determine the one or more landmark profiles most similar to said first or predicted profile, each landmark profile comprising measured amounts of a plurality of cellular constituents in a second cell of said cell type or type of organism having a perturbation to a known gene or its encoded RNA or protein, wherein the genes, or their encoded RNAs or proteins, perturbed in the one or more landmark profiles determined in step (b) are those candidate genes responsible for the phenotype of interest.

In a second embodiment, the present invention relates to a method for determining one or more candidate genes, or their encoded RNAs or proteins, responsible for a phenotype of interest displayed by a cell type or organism, comprising comparing a first profile or a predicted profile derived therefrom to a database comprising a plurality of landmark profiles to determine the one or more landmark profiles most similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and wherein the genes, or their encoded RNAs or proteins, perturbed in the one or more landmark profiles determined to be most similar are those candidate genes responsible for the phenotype of interest.

In a third embodiment, the present invention relates to a method for relating the phenotype of a cell type or organism to a genotype, said method comprising: (a) determining measured amounts of a plurality of cellular constituents in a first cell of said cell type or of said organism exhibiting a phenotype, to create a first profile; (b) determining measured amounts of a plurality of cellular constituents in a second cell of said cell type or of said organism having a genetic perturbation to a known gene to create a landmark profile; and (c) determining the degree of similarity between said first profile and said landmark profile by comparing said degree of similarity between the measured amounts determined for said pluralities of cellular constituents, wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

In a fourth embodiment, the present invention relates to a method of determining if a genotype associated with a phenotype of interest is present in a cell type or organism, comprising: (a) determining measured amounts of a plurality of cellular constituents in a first cell of said cell type or organism to create a first profile; and (b) comparing said first profile to a database of a plurality of landmark profiles to determine whether one or more landmark profiles known to be indicative of the presence or absence of a genotype associated with the phenotype of interest is similar to said first profile, each landmark profile comprising measured amounts of a plurality of cellular constituents in a second cell of said cell type or type of organism having a perturbation to a known gene or its encoded RNA or protein, wherein determining that the landmark profiles known to be indicative of the presence of said genotype are similar to said first profile, is indicative of the presence of said genotype associated with the phenotype of interest in the cell type or organism; and wherein determining that the landmark profiles known to be indicative of the absence of said genotype are similar to said first profile, is indicative of the absence of said genotype associated with the phenotype of interest in the cell type or organism.

In a fifth embodiment, the present invention relates to a method of determining if a genotype associated with a phenotype of interest is present in a cell type or organism, comprising comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

In a sixth embodiment, the present invention relates to a system for determining one or more candidate genes, or their encoded RNAs or proteins, responsible for a phenotype of interest displayed by a cell or organism, said system comprising: (a) one or more memory units; and (b) one or more processor units interconnected with the one or more memory units, wherein the one or more memory units encodes one or more programs causing the one or more processor units to perform a method comprising comparing a first profile or a predicted profile derived therefrom to a database comprising a plurality of landmark profiles to determine the one or more landmark profiles most similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and wherein the genes perturbed in the one or more landmark profiles determined to be most similar are those candidate genes responsible for the phenotype of interest.

In a seventh embodiment, the present invention relates to a system for relating the phenotype of a cell type or organism to a genotype, said system comprising: (a) one or more memory units; and (b) one or more processor units interconnected with the memory, wherein the one or more memory units encodes one or more programs causing the one or more processor units to perform a method comprising determining the degree of similarity between a first profile of a plurality of cellular constituents in a first cell of said cell type or of said organism exhibiting a phenotype and a landmark profile of a plurality of cellular constituents in a second cell of said cell type or of said organism having a genetic perturbation to a known gene by comparing said degree of similarity between the measured amounts of said pluralities of cellular constituents, wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

In a eighth embodiment, the present invention relates to a system for determining if a genotype associated with a phenotype of interest is present in a cell type or organism, said system comprising: (a) one or more memory units; and (b) one or more processor units interconnected with the one or more memory units, wherein the one or more memory units encodes one or more programs causing the one or more processor units to perform a method comprising comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

In a ninth embodiment, the present invention relates to a computer program product for use in conjunction with a computer having one or more memory units and one or more processor units, the computer program product comprising a computer readable storage medium having a computer program mechanism encoded thereon, wherein said computer program mechanism may be loaded into the one or more memory units of a computer and cause the one or more processor units of the computer to execute the step of comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

In a tenth embodiment, the present invention relates to a method for relating the phenotype of a cell type or organism to a genotype, said method comprising: determining the degree of similarity between a first profile and a landmark profile by comparing the degree of similarity between measured amounts of pluralities of cellular constituents, wherein said first profile comprises measured amounts of a plurality of cellular constituents in a first cell of said cell type or of said organism exhibiting a phenotype, and wherein said landmark profile comprises measured amounts of a plurality of cellular constituents in a second cell of said cell type or of said organism having a genetic perturbation to a known gene, wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

### 4. BRIEF DESCRIPTION OF THE DRAWINGS

FIG. 1 illustrates transcriptional response space inhabited by meausurements of phenotypes and genetic landmarks.

FIG. 2(a-f) illustrates the correlation between the transcriptional response profile of yeast treated with clotrimazole and the transcriptional response profiles of yeast with perturbations in SWI4, RPD3, CNA1 CNA2, HMG2 and ERG11 genes. A straight line (higher correlation coefficient, r) indicates the greatest similarity.

FIG. 3 illustrates transcriptional profiles for a set of 300 landmark profiles, 276 of which were deletion mutant yeast strains, 13 of which were drug treatments using well-characterized compounds and 11 of which strains contain under-expression alleles of genes that reduce expression of a given known gene. Data were clustered using methods described herein, using genes and experiments that fulfilled the following criteria: P<0.01, log₁₀(ratio)>0.5 and genes and experiments in at least two experiments.

FIG. 4 illustrates a computer system useful for embodiments of the invention.

### 5. DETAILED DESCRIPTION OF THE INVENTION

This section presents a detailed description of the invention and its applications. This description is by way of several exemplary illustrations, in increasing detail and specificity, of the general methods of this invention. These examples are non-limiting, and related variants will be apparent to one of skill in the art.

Although, for simplicity, this disclosure often makes references to gene expression profiles, transcript levels, etc., in yeast, it will be understood by those skilled in the art that the methods of the invention are useful for the analysis of any biological response profile in any organism, and are particularly well-suited to monitor genetic engineering and selective cross-breeding in crops and livestock. In particular, one skilled in the art will recognize that the methods of the present invention are equally applicable to biological profiles that comprise measurements of other cellular constituents such as, but not limited to, measurements of protein abundance or protein activity levels.

Moreover, although for simplicity this disclosure often makes reference to “a cell” (e.g., “mutation of a gene in a cell”), it will be understood by those of skill in the art that any particular step of the invention will also be construed as covering use of a plurality of cells, e.g., from a tissue sample from an organism, or from a cultured cell line. A “cell type,” as used herein, can refer to a cell of a species of interest (e.g., corn, bean, human, mouse), a lineage of interest (e.g., blood cell, nerve cell, skin cell), or a tissue of interest (e.g., lung, brain, heart). Such cells can be from naturally single-celled organisms or derived from multi-cellular higher organisms. The cell can be a cell of a plant or an animal (including but not limited to mammals, primates, humans, and non-human animals such as dogs, cats, horses, cows, sheep, mice, rats, etc.)

The methods of the present invention may be applied to any organism, but are particularly well-suited to analysis of crops and livestock. Crop plants suitable for analysis by the methods of the present invention include, but are not limited to, corn, wheat, rice, barley, oats, hops, rye, millet, soy beans, alfalfa, cotton, tobacco, sugarcane, hemp and sugarbeets. Livestock animals suitable for analysis by the methods of the present invention include, but are not limited to, cattle, sheep, goats, pigs, horses, buffalo, alpaca, llamas, and poultry.

### 5.1 Introduction

A mutation of a gene in a cell may have effects on the biological state of a cell, which can be represented by measured amounts of cellular constituents as defined in Section 5.1.1, below. The altered genotype of a cell, in addition to affecting the biological state of the cell, may also affect the phenotype. Accordingly, one aspect of the present invention provides methods for relating the biological state of a cell to genotype and phenotype. This invention is partially premised upon a discovery of the inventors that the biological state of a cell with a particular phenotype can be compared to the biological states of cells with known genotypes (mutations in known genes), thereby indicating the genes or biochemical pathways involved in creating the phenotype. The invention is also partially premised upon the inventors' discovery that measured amounts of a plurality of cellular constituents of a cell or organism can be used as the phenotypic marker of a particular genotype.

This section first presents a background about representations of biological state and biological responses in terms of measured amounts of cellular constituents. Next, a schematic and non-limiting overview of the invention is presented, and the representation of biological states and biological responses according to the method of this invention is introduced. The following sections present specific non-limiting embodiments of this invention in greater detail.

### 5.1.1 Definition of Biological State

The effects of a genetic mutation are detected in the instant invention by measurements and/or observations made on the biological state of a cell. The biological state of a cell, as used herein, is taken to mean the state of a collection of cellular constituents, including but not limited to RNA abundances, protein abundances, and protein activities, which are sufficient to characterize the cell for an intended purpose, such as for characterizing the effects of a genetic mutation. As used herein, the term “cellular constituents” is not intended to refer to known subcellular organelles, such as mitochondria, lysozomes, etc. The measurements and/or observations made on the state of these constituents can be of their abundances (i.e., amounts or concentrations in a cell), or their activities, or their states of modification (e.g., phosphorylation), or other measurement relevant to the characterization of genetic mutations. In various embodiments, this invention includes making such measurements and/or observations on different collections of cellular constituents. These different collections of cellular constituents are also called herein aspects of the biological state of the cell.

One aspect of the biological state of a cell usefully measured in the present invention is its transcriptional state. The transcriptional state is the currently preferred aspect of the biological state measured in this invention. The transcriptional state of a cell is the identities and abundances of the constituent RNA species, especially mRNAs, in the cell under a given set of conditions. Preferably, a substantial fraction of all constituent RNA species in the cell are measured, but at least, a sufficient fraction is measured to characterize the action of a genetic mutation of interest. It can be conveniently determined by, e.g., measuring cDNA abundances by any of several existing gene expression technologies. One particularly preferred embodiment of the invention employs DNA arrays for measuring mRNA or transcript levels of a large number of genes.

Another aspect of the biological state of a cell usefully measured in the present invention is its translational state. The translational state of a cell is defined herein to be the identities and abundances of the constituent protein species in the cell with a specific genetic mutation. Preferably, a substantial fraction of all constituent protein species in the cell are measured, but at least, a sufficient fraction is measured to characterize the genetic mutation of interest. The transcriptional state of a cell can often be used as a representative of the translational state of a cell.

Other aspects of the biological state of a cell are also of use in this invention. For example, the activity state of a cell, as that term is used herein, refers to the activities of the constituent protein species (and also optionally catalytically active nucleic acid species) in the cell under a given set of conditions. The translational state of a cell can often be used as a representative of the activity state of a cell.

This invention is also adaptable, where relevant, to “mixed” aspects of the biological state of a cell in which measurements of different aspects of the biological state of a cell are combined. For example, in one mixed aspect, the abundances of certain RNA species and of certain protein species, are combined with measurements of the activities of certain other protein species. Further, it will be appreciated from the following that this invention is also adaptable to other aspects of the biological state of the cell that are measurable.

The biological state of a cell can be represented by a profile of some number of cellular constituents. Such a profile of cellular constituents can be represented by the vector S.

S=[S₁, . . . Sᵢ, . . . Sₖ] (1)

Where Sᵢ is the level of the i'th cellular constituent, for example, the transcript level of gene i, or alternatively, the abundance or activity level of protein i.

In some embodiments, cellular constituents are measured as continuous variables. For example, transcriptional rates are typically measured as number of molecules synthesized per unit of time. Transcriptional rate may also be measured as percentage of a control rate. However, in some other embodiments, cellular constituents may be measured as categorical variables. For example, transcriptional rates may be measured as either “on” or “off”, where the value “on” indicates a transcriptional rate above a predetermined threshold and value “off” indicates a transcriptional rate below that threshold.

### 5.1.2 Representation of Biological Responses

The responses of a cell to a genetic mutation can be measured by observing the changes in the biological state of the cell. A response profile is a collection of changes of cellular constituents. In the present invention, the response profile of a cell to the perturbation m is defined as the vector v^{(m)}:

v^{(m)}=[v₁^{(m)}, . . . vᵢ^{(m)}, . . . vₖ^{(m)}] (2)

Where vᵢ^{m} is the amplitude of response of cellular constituent i under the perturbation m. In some particularly preferred embodiments of this invention, the biological response to a genetic mutation is measured by the induced change in the transcript level of at least 2 genes, preferably more than 10 genes, more preferably more than 100 genes and most preferably more than 1,000 genes.

In some embodiments of the invention, the response is simply the difference between biological variables in a wild-type cell and a mutated cell. In some preferred embodiments, the response is defined as the ratio or the logarithm of the ratio of cellular constituents of a wild-type cell and a mutated cell, and is called an expression ratio.

In some preferred embodiments, vᵢ^{m} is set to zero if the response of gene i is below some threshold amplitude or confidence level determined from knowledge of the measurement error behavior. In such embodiments, those cellular constituents whose measured responses are lower than the threshold are given the response value of zero, whereas those cellular constituents whose measured responses are greater than the threshold retain their measured response values. This truncation of the response vector is a good strategy when most of the smaller responses are expected to be greatly dominated by measurement error. After the truncation, the response vector v^{(m)} also approximates a ‘matched detector’ (see, e.g., Van Trees, 1968, Detection, Estimation, and Modulation Theory Vol. I, Wiley & Sons) for the existence of genetic mutations affecting similar pathways. It is apparent to those skilled in the art that the truncation levels can be set based upon the purpose of detection and the measurement errors. For example, in some embodiments, genes whose transcript level changes are lower than two fold or more preferably four fold are given the value of zero.

### 5.2 Methods for Relating Phenotype to Genotype

This section presents first the general methods of this invention, then presents certain alternative embodiments of the invention, and finally presents applications of the methods of the invention to genetic interpretation and to prediction of crop and livestock phenotypes.

### 5.2.1 General Methods of the Invention

The methods of this invention employ certain types of cells, certain observations of changes in aspects of the biological state of a cell, and certain comparisons of these observed changes. In the following, these cell types, observations, and comparisons are described in turn in detail.

The present invention makes use of two principal types of cells: wild-type cells, and modified cells. “Wild-type” cells are reference, or standard, cells used in a particular application or embodiment of the methods of this invention. Being only a reference cell, a wild-type cell, need not be a cell normally found in nature, and often will be a recombinant or genetically altered cell line. Usually the cells are cultured in vitro as a cell line or strain. Other cell types used in the particular application of the present invention are preferably derived from the wild-type cells. Less preferably, other cell types are derived from cells substantially isogenic with wild-type cells. For example, wild-type cells might be a particular cell line of the yeast Saccharomyces cerevisiae, or a particular mammalian cell line (e.g., HeLa cells). Although, for simplicity this disclosure often makes reference to single cells (e.g., “RNA is isolated from a cell deleted for a single gene”), it will be understood by those of skill in the art that more often any particular step of the invention will be carried out using a plurality of genetically identical cells, e.g. from a cultured cell line.

Two cells are said to be “substantially isogenic” where their expressed genomes differ by a known amount that is preferably at less than 10% of genetic loci, more preferably at less that 1%, or even more preferably at less than 0.1%. Alternately, two cells can be considered substantially isogenic when the portions of their genomes relevant to the effects of a drug of interest differ by the preceding amounts. It is further preferable that the differing loci be individually known.

“Modified cells” are derived from wild-type cells by modifications to the genome of the wild-type cells. As is commonly appreciated, protein activities result in part from protein abundances; protein abundances result from translation of mRNA (balanced against protein degradation); and mRNA abundances result from transcription of DNA and splicing of mRNA precursors (balanced against mRNA degradation). Therefore, genetic level modifications to a cellular DNA constituent alters transcribed mRNA abundances, translated protein abundances, and ultimately protein activities. Two types of modified wild-type cells of particular interest are deletion mutants and over-expression mutants. Deletion mutants are wild-type cells that have been modified genetically so that a single gene, usually a protein-coding gene, is substantially deleted. As used herein, deletion mutants also include mutants in which a gene has been disrupted so that usually no detectable mRNA or bioactive protein is expressed from the gene, even though some portion of the genetic material may be present. In addition, in some embodiments, mutants with a deletion or mutation that removes or inactivates one activity of a protein (often corresponding to a protein domain) that has two or more activities, are used and are encompassed in the term “deletion mutants.” Over-expression mutants are wild-type cells that are modified genetically so that at least one gene, most often only one, in the modified cell is expressed at a higher level as compared to a cell in which the gene is not modified (i.e., a wild-type cell). Alternatively and less preferably, the deletion and over-expression mutants may not be derived from the wild-type cells but may instead be derived from cells that are substantially isogenic with wild-type cells, except for their particular genetic modifications.

The method of the invention involves observing changes in any of several aspects of the biological state of a cell (e.g., changes in the transcriptional state, in the translational state, in the activity state, and so forth) between a wild-type cell and a cell with a genetic mutation. A relative increase or decrease e.g., in response to a genome modification, in the amount of a cellular constituent measured in an aspect of the biological state of the cell (e.g., specific mRNA abundances, protein abundances, protein activities, levels of modification and so forth) is called a perturbation. An increase is called a positive perturbation, and a decrease a negative perturbation. No significant detectable change is called no perturbation. By way of example, a “perturbation” can be achieved by introducing one or more point mutations, insertions, or deletions into the gene of interest, or by over-expression or under-expression of its encoded RNA or protein (see Section 5.3 and its subsections, infra). The set of perturbations observed for cellular constituents (including, optionally, cellular constituents with no perturbation) can be referred to as a perturbation pattern or a perturbation array or, more preferably, a profile. Depending on the measurement techniques, perturbations may be scored qualitatively simply as a positive, a negative, or no perturbation, or actual quantitative values may be available and compared. For example, a profile can be a pattern of changes in mRNA abundances, protein abundances, protein activity levels, or so forth.

As used herein, a first cellular constituent and a second cellular constituent (that are the same or different and are from the same or a different cell) are said to be “differently perturbed” when, for the first cellular constituent, there is a positive perturbation, and for the second cellular constituent there is no perturbation or a negative perturbation. In addition, the two cellular constituents are said to be “differently perturbed” if, for the first cellular constituent there is a negative perturbation and for the second cellular constituent there is no perturbation or a positive perturbation. Furthermore, two cellular constituents are said to be “differently perturbed” if for the first cellular constituent there is no perturbation, and for the second cellular constituent there is a positive perturbation or a negative perturbation. In cases where the values of perturbations are measured, two perturbation can be said to be “differently perturbed” where the measured values for the two perturbations are detectably different, preferably having a statistically significant difference. As used herein, perturbations of a first and a second cellular constituent are said to be the “same” when both have a negative or a positive perturbation, or where the measured values are not significantly different.

The actual values present in a profile depend essentially on the measurement methods available for the particular cellular constituents being measured. Where quantitative abundances or activities are available, either in absolute or relative units, a numerical abundance or activity ratio can be calculated and placed in the profile. For example, in the case of transcriptional state measurements by quantitative gene expression technologies, a numerical expression ratio of the abundances of cDNAs (or mRNAs in an appropriate technology) in the two states can be calculated. Alternatively, a logarithm (e.g., log₁₀) (or another monotonic function) of the abundance ratio can be used. Where only qualitative data is available, arbitrary integer values can be assigned to each type of perturbation of a cellular constituent. For example, the value +1 can be assigned to a positive perturbation; the value −1 to a negative perturbation; and the value 0 to no perturbation.

It is often convenient to represent graphically a profile as a two-dimensional physical array of perturbation values. When making such a graphical representation, the assignment of particular perturbation values to particular array positions can be entirely arbitrary or can be guided by any convenient principles. For example, related cellular constituents, such as genes, proteins, or protein activities of a particular pathway, can be grouped together, e.g., by “clustering” as described in co-pending U.S. patent application Ser. Nos. 09/220,142 (filed Dec. 23, 1998) and 09/428,427 (filed Oct. 27, 1999), which are incorporated herein by reference in their entirety. In the case of transcriptional state measurements by gene transcript arrays, the transcript profile can be arranged as the transcript array is arranged.

In preferred embodiments, the effects of a genetic mutation are determined by observing and comparing changes in the transcriptional state of a cell. Although homeostatic mechanisms in cells are not limited to transcriptional controls, analysis of the transcriptional state is often found sufficient for purposes of characterizing a genetic mutation. First, most genetic mutations produce a significant and characteristic change in the transcriptional state of the cell. Second, because homeostatic control mechanisms acting at a variety of levels in cells generally appear to move in the same direction, corresponding cellular constituents at the transcriptional level, the translational level, and the activity level often change in the same direction. For example, the down regulation of cyclin transcription in yeast is accompanied by cyclin inactivation by phosphorylation and degradation by ubiquitin-mediated proteolysis Nasmyth, 1996, At the heart of the budding yeast cycle, TIG 12:405-412). Thus, a cellular response that activates (or inhibits) the activity or prevalence of a given protein at one level is often accompanied by a corresponding transcript induction (or reduction) response.

The modified-cell profile includes a plurality of perturbation values that represent the perturbation in cellular constituents observed in an aspect of the biological state of a modified cell resulting from an indicated gene deletion. An aspect of the biological state of a modified cell with a genetic mutation is measured and compared to that aspect of the biological state of the cell without such a mutation (wild-type) in order to determine the cellular constituents in this aspect that are perturbed or are not perturbed. Such a profile is not generally limited to revealing only changes directly due to the mutation, because changes in the elements of the biological state that are indirectly affected by the particular mutation or its products will also be apparent. This type of profile provides information about the effects of the mutated gene on the biological state of a wild-type cell. The plurality of perturbations comprises at least five different perturbations, preferably at least ten different perturbations, more preferably at least 50 different perturbations, and most preferably at least 100 different perturbations. The methods of this invention compare these effects to the effects that result in a cell having a particular phenotype. A group of these profiles, e.g., for known point mutations, insertions, deletions, over-expression, under-expression, etc., in particular genes (called herein a compendium of landmark profiles) is assembled for relating genotype to phenotype.

A perturbation to a known gene can be by virtue of not only insertions, deletions, point mutations, etc. that have been mapped to a specific location within a gene, but also one or more mutations in a gene that have not yet been mapped. Thus, a random insertion mutant that is profiled, but wherein the mutation has not yet been mapped, may be an example of a cell or organism having a perturbation to a known gene.

It will be understood that landmark profile that is “indicative of the presence or absence of a genotype”, as used herein, does not have to conclusively indicate that a genotype is present or absent. A landmark profile that is said to be indicative of the presence or absence, respectively, of a genotype indicates an increased probability that the genotype is present or absent, respectively, which can be with varying degrees of certainty, from the genotype being more likely than not present or absent, to it being reasonably conclusive that the genotype is present or absent, respectively.

In a specific embodiment, in which the observed aspect of the biological state is the transcriptional state, and in which the transcriptional state is measured by hybridization to a gene transcript array, these transcript profiles are measured in the following ways. The modified-cell profile is determined by observing the mutant transcript array. In particular, deletion transcript profiles, where the genome modification includes gene deletion, and over-expression transcript profiles, where the genome modification includes gene over-expression, are examples of mutant transcript profiles. Even where the transcriptional state is measured by other gene expression technologies, it can be convenient to refer to these profiles as “transcript profiles.”

In view of the previously described cell types, perturbations, and profiles, the methods for relating genotype to phenotype according to the present invention identify the probable genotype that causes the appearance of a particular phenotype by measuring and comparing profiles. In one preferred general embodiment, the methods include two principal steps. The first step includes determining measured amounts of (i.e., measuring) a plurality of cellular constituents to obtain a profile of a modified cell having a desired phenotype. In one embodiment, when the transcriptional state is observed, the cellular constituents are mRNA species and perturbations to the measured amounts of cellular constituents are represented by relative increases or decreases in measured amounts of mRNA species (e.g., compared to a wild-type cell). In another embodiment, the transcriptional state may be related to the absolute measured amounts (abundances or activities) of cellular constituents, e.g., the number of, for example, mRNA molecules, in a cell. Alternatively, when the translational state is observed, the cellular constituents are protein species, and the perturbation may be a change in the measured amounts of protein species. In yet another embodiment, a combination of the transcriptional and translational states of a cell type is observed. Alternatively, where a profile of a modified cell is already available, the first step of measuring cellular constituents to obtain the profile can be omitted.

The second step includes comparing the profile of a modified cell having a desired phenotype to a database of landmark profiles each of which arises from a modified cell having an indicated genetic mutation (i.e. a compendium) to determine the degree of similarity between the profile of a modified cell and the landmark profiles. When the transcriptional state is observed in the first step, the profile is preferably compared to a compendium comprising landmark profiles generated from measurements of the transcriptional state of modified cells with indicated genetic mutations. Preferably, this compendium is a compendium of deletion transcript profiles, in which each deletion transcript profile depicts the transcriptional state of a cell in which a single gene has been disrupted. The deletion profiles having the greatest similarity to the modified cell profile indicate which genes are involved in biological pathways responsible for the desired phenotype of the modified cell.

In another embodiment, amounts of a plurality of cellular constituents are measured in a cell of a cell type, and a predicted profile is derived therefrom for comparison to one or more landmark profiles. The predicted profile may be for different cellular constituents than those for which amounts were measured in the experiment. For example, a translational profile of protein levels may be used to predict the corresponding transcript profile, which may be used for comparison to a database comprising landmark transcript profiles. Alternatively, an expression profile of an immature organism, e.g. a seedling, may be acquired and may be used to predict an expression profile of the mature organism.

In yet another embodiment, the measured amounts of cellular constituents comprising an expression profile in a modified cell type are not compared to the measured amounts of cellular constituents of a wild-type cell of that cell type. Rather, the expression profile comprises absolute measured amounts of cellular constituents, e.g., abundance of mRNA, for example.

The identity of the cellular constituents for which measured amounts are present in each of the landmark profiles and in the profiles in the various steps of the invention are preferably the same but need not be, as long as there is overlap in the cellular constituents.

### 5.2.2 Alternative Embodiments

This subsection describes alternative embodiments relating to the use of compendiums for relating genotype to phenotype.

In one alternative embodiment, the phenotype of a modified cell can be predicted based on the profile of the modified cell. A profile of a modified cell of unknown phenotype is compared to a compendium comprising landmark profiles, some of which are associated with known phenotypes, to determine the degree of similarity between the profile of the modified cell and the landmark profiles. The profile of the modified cell comprises measured amounts of a plurality of cellular constituents, some of which are perturbed in the cell's modified state. Phenotype(s) associated with landmark profile(s) having the greatest similarity to the modified cell profile predict the phenotype of the modified cell.

In a second alternative embodiment, the phenotype of a mature modified cell can be predicted from the profile of the immature modified cell. A profile of an immature modified cell is compared to a compendium comprising landmark profiles each of which arises from an immature modified cell having an indicated perturbation to determine the degree of similarity between the profile of the immature modified cell and the landmark profiles. Similarity of the immature profiles indicates eventual similarity of mature profiles. By comparing the profile of an immature modified cell to the landmark profiles of immature modified cells with indicated genetic mutations, causative genes of the phenotype of the immature cell can be identified and the eventual phenotype of the mature cell can be inferred.

### 5.3 Generation of a Compendium of Landmark Profiles

In a preferred embodiment, the biological state of a cell is determined by measuring the expression levels of a plurality of genes in a cell to produce a transcript profile. The effects of mutations of individual genes in a cell can be conveniently and exhaustively examined by using a library of cell mutants, wherein each mutant has been modified at a different genetic locus by techniques including, but not limited to, transfection, homologous recombination, promoter replacement, or RNA anti-sense approaches. The transcript profiles of each of these mutant cells are measured to produce a “compendium” comprising landmark transcript profiles, each of which is uniquely associated with a mutation in a particular gene of the organism. One of ordinary skill in the art will readily recognize that a compendium can also be constructed by measuring other cellular constituents that are indicative of the biological states of mutant cells, which include, but are not limited to, protein expression and protein activity levels. Preferably, the compendium comprising landmark profiles is a database stored on a computer that carries out the comparisons. In specific embodiments, the database contains at least 10 profiles, at least 50 profiles, at least 100 profiles, at least 500 profiles, at least 1,000 profiles, at least 10,000 profiles, or at least 50,000 profiles, each profile containing measurements of at least 10, preferably at least 50, more preferably at least 100, more preferably at least 500, even more preferably at least 1,000, even more preferably at least 10,000, most preferably at least 50,000 cellular constituents.

In some embodiments, a library of mutants is generated by targeting mutations to particular genes of an organism. As shown in the Example, below, Saccharomyces cerevisiae is particularly well-suited to this technique of generating mutants. While many organisms repair double-stranded DNA ends that are not part of telomeres by end-to-end ligation, S. cerevisiae uses homologous recombination. Thus, targeted perturbations of genes can be made in yeast by transforming the yeast with a particular DNA sequence, which integrates at a locus with high homology. In other embodiments, a library of mutants is generated by random mutagenesis using, e.g., chemical agents, radiation or retroviral-mediated insertion mutagenesis and subsequent location of the mutation in the genome of the organism.

One of ordinary skill in the art will recognize that biological state profiles may change with environmental perturbations, so that when generating a compendium comprising landmark profiles, differences in environmental variables, e.g., growth medium, temperature, cell density, pH, etc., should be minimized. Likewise, when comparing a new transcript profile to the compendium, the organism or cell from which that profile was generated should be grown under the same environmental conditions as the mutants from which the compendium was compiled. One of ordinary skill in the art will further recognize that, in the case of multicellular organisms, profiles will change with tissue type and developmental state.

In one embodiment, the database comprises landmark profiles for perturbations to at least 2%, preferably at least 5%, more preferably at least 20%, even more preferably at least 15%, even more preferably at least 40%, most preferably at least 75%, of genes in the genome of a cell type or organism, and may also include profiles from over-expression and under-expression strains, since these will be fundamentally different from profiles of complete gene deletions. In another embodiment, the number of landmark profiles is reduced to the minimum necessary to identify genes that cause a desired set of phenotypes. For example, genetic perturbations that are expected to have similar transcriptional effects can be represented by only one profile of one disruption in the compendium set, i.e., each known biological pathway can be represented in the compendium by at least one profile of one perturbation, but multiple perturbations from each pathway may not be necessary.

In a specific embodiment, the database comprises landmark profiles for perturbations to at least 100, preferably at least 250, more preferably at least 500, even more preferably at least 1,000, even more preferably at least 10,000, even more preferably at least 50,000, most preferably at least 100,000 genes in the genome of a cell or organism. In another embodiment, the database comprises landmark profiles for perturbations to at least ¼, preferably at least ½, most preferably at least ¾ of the genes in the genome of a cell or organism. In various embodiments, the cell or organism for which the database contains landmark profiles is a human, livestock animal or plant.

### 5.3.1 Genetic Modifications

Genetically modified cells, i.e., mutant cells, can be made using cells of any organism for which genomic sequence information is available and for which methods are available that allow underexpression (including complete deletion) of specific genes, or over-expression of specific genes. The genetically modified cells are used to make mutant transcript profiles. Preferably, a compendium is constructed that includes transcript profiles that represent the transcriptional states of each of a plurality of modified cells with an indicated genetic mutation, e.g., a set of cells in which each cell is genetically modified. Such a compendium is advantageous to relate genotype to phenotype in a systematic and automatable manner. Preferably, the compendium includes mutant transcript profiles for the genes likely to be involved in biological pathways that are responsible for producing a desired phenotype. Systematic efforts to create large collections of mapped insertion mutants is underway for several eukaryotic organisms, including nematodes (Pennisi (1998) Science 282:1972-74), plants (e.g., Arabidopsis, Somerville et al. (1999) Science 285:380-383), and flies (e.g., D. melanogaster, Spradling et al. (1999) Genetics 153:135-177).

In one embodiment, the invention is carried out using a yeast, with Saccharomyces cerevisiae most preferred because the sequence of the entire genome of a S. cerevisiae strain has been determined. In addition, well-established methods for deleting or otherwise disrupting or modifying specific genes are available in yeast. It is believed that most (approximately four-fifths) of the genes in S. cerevisiae can be deleted, one at a time, with little or no effect on the ability of the organism to reproduce. Another advantage is that biological functions are often conserved between yeast and humans. For example, almost half of the proteins identified as defective in human heritable diseases show amino acid similarity to yeast proteins (Goffeau et al., 1996, Life with 6000 genes. Science 274:546567). A preferred strain of yeast is a S. cerevisiae strain for which yeast genomic sequence is known, such as strain S288C or substantially isogenic derivatives of it (see, e.g., Nature 369, 371-8 (1994); P.N.A.S. 92:3809-13 (1995); E.M.B.O.J. 13:5795-5809 (1994), Science 265:2077-2082 (1994); E.M.B.O.J. 15:2031-49 (1996), all of which are incorporated herein. However, other strains may be used as well. Yeast strains are available from American Type Culture Collection, Rockville, Md. 20852. Standard techniques for manipulating yeast are described in C. Kaiser, S. Michaelis, & A. Mitchell, 1994, Methods in Yeast Genetics: A Cold Spring Harbor Laboratory Course Manual, Cold Spring Harbor Laboratory Press, New York; and Sherman et al., 1986, Methods in Yeast Genetics: A Laboratory Manual, Cold Spring Harbor Laboratory, Cold Spring Harbor. N.Y., both of which are incorporated by reference in their entirety and for all purposes.

### 5.3.2 Construction of Deletion and Over-Expression Mutants in Yeast

In one embodiment of the invention, yeast cells are used. In one embodiment, yeast genes are disrupted or deleted using the method of Baudin et al., 1993, A simple and efficient method for direct gene deletion in Saccharomyces cerevisiae, Nucl. Acids Res. 21:3329-3330, which is incorporated by reference in its entirety for all purposes. This method uses a selectable marker, e.g., the KanMx gene, which serves in a gene replacement cassette. The cassette is transformed into a haploid yeast strain and homologous recombination results in the replacement of the targeted gene (ORF) with the selectable marker. In one embodiment, a precise null mutation (a deletion from start codon to stop codon) is generated. Also see, Wach et al., 1994, New heterologous modules for classical or PCR-based gene perturbations in Saccharomyces cerevisiae, Yeast 10:1793-1808; Rothstein, 1991, Methods Enzymol. 194:281 each of which is incorporated by reference in its entirety for all purposes. An advantage to using precise null mutants is that it avoids problems with residual or altered functions associated with truncated products. However, in some embodiments (e.g., when investigating potential targets in the excluded set, Section 5.6, infra) a deletion or mutation affecting less than the entire protein coding sequence, e.g., a deletion of only one domain of a protein having multiple domains and multiple activities, is used.

In some embodiments, the polynucleotide (e.g., containing a selectable marker) used for transformation of the yeast includes an oligonucleotide marker that serves as a unique identifier of the resulting deletion strain as described, for example, in Shoemaker et al., 1996, Nature Genetics 14:450. Once made, perturbations can be verified by PCR using the internal KanMx sequences, or using an external primer in the yeast genome that immediately flanks the disrupted open reading frame, and assaying for a PCR product of the expected size. When yeast is used, it may sometimes be advantageous to disrupt ORFs in three yeast strains, i.e., haploid strains of the a and α mating types, and a diploid strain (for deletions of essential genes).

In another embodiment, precise deletion of yeast genes is accomplished by using a PCR-mediated gene disruption strategy using homologous recombination (Winzeler et al. (1999) Science 285:901-906). In this method, short regions of yeast sequence that are upstream and downstream of a targeted gene are placed at each end of a selectable marker gene through PCR. The resulting PCR products, when transformed into yeast, can replace the targeted gene by homologous recombination. For most genes, greater than 95% of the yeast transformants carry the correct gene deletion.

Over-expression mutants are preferably made by modifying the promoter for the gene of interest, usually by replacing the promoter with a promoter other than that naturally associated with the gene, such as an inducible promoter. In addition, or alternatively, an enhancer sequence can be added or modified. Other methods for carrying out genetic modification to increase expression from a predetermined gene are well known in the art, and include expression from vectors, such as plasmids, carrying the gene of interest.

### 5.3.3 Construction of Mutants in Other Organisms

The method of the present invention can be carried out using cells from any eukaryote for which genomic sequence of at least one gene is available, e.g., fruit flies (e.g., D. melanogaster), nematodes (e.g., C. eleganis), and mammalian cells such as cells derived from mice and humans. For example, 100% of the genome of D. melanogaster has been sequenced (Jasny, 2000, Science 287:2181). Methods for disruption of specific genes are well known to those of skill in the art, see, e.g., Anderson, 1995, Methods Cell Biol. 48:31; Pettitt et al., 1996, Development 122:4149-4157; Spradling et al., 1995, Proc. Natl. Acad. Sci. USA; Ramirez-Solis et al., 1993, Methods Enzymol. 225:855; and Thomas et al., 1987, Cell 51:503, each of which is incorporated herein by reference in its entirety for all purposes.

Other known methods of cellular modification target RNA abundances or activities, protein abundances, or protein activities. Examples of such methods are described in the following.

Methods of Modifying RNA Abundances or Activities

Methods of modifying RNA abundances and activities currently fall within three classes, ribozymes, antisense species, and RNA aptamers (Good et al., 1997, Gene Therapy 4: 45-54). Ribozymes are RNAs which are capable of catalyzing RNA cleavage reactions. (Cech, 1987, Science 236:1532-1539; PCT International Publication WO 90/11364, published Oct. 4, 1990; Sarver et al., 1990, Science 247: 1222-1225). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA (Haseloff et al., 1988, Nature 334:585-591; Koizumi et al., 1988, FEBS Lett., 228:228-230; Koizmi et al., 1988, FEBS Lett., 239:285-288). Ribozyme methods involve exposing a cell to, inducing expression in a cell, etc. of such small RNA ribozyme molecules. (Grassi and Marini, 1996, Annals of Medicine 28: 499-510; Gibson, 1996, Cancer and Metastasis Reviews 15: 287-299).

Ribozymes can be routinely expressed in vivo in sufficient number to be catalytically effective in cleaving mRNA, and thereby modifying mRNA abundances in a cell. (Cotten et al., 1989, Ribozyme mediated destruction of RNA in vivo, The EMBO J. 8:3861-3866). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. tDNA genes (i.e., genes encoding tRNAs) are useful in this application because of their small size, high rate of transcription, and ubiquitous expression in different kinds of tissues. Alternately, an inducible promoter (e.g., a glucocorticoid or a tetracycline response element) can by used so that ribozyme expression can be selectively controlled. Therefore, ribozymes can be routinely designed to cleave virtually any mRNA sequence, and a cell can be routinely transformed with DNA coding for such ribozyme sequences such that a catalytically effective amount of the ribozyme is expressed. Accordingly the abundance of virtually any RNA species in a cell can be essentially eliminated.

In another embodiment, activity of a target RNA (preferable mRNA) species, specifically its rate of translation, is inhibited by use of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered to a cell or which can be produced intracellularly by transcription of exogenous, introduced sequences in quantities sufficient to inhibit translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO 88/09810, published Dec. 15, 1988), hybridization-triggered cleavage agents (see, e.g., Krol et al., 1988, BioTechniques 6: 958-976) or intercalating agents (see, e.g., Zon, 1988, Pharm. Res. 5: 539-549).

In a preferred aspect of the invention, an antisense oligonucleotide is provided, preferably as single-stranded DNA. The oligonucleotide may be modified at any position on its structure with constituents generally known in the art.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15: 6625-6641).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc. In another embodiment, the oligonucleotide is a 2′-0-methyhibonucleotide (Inoue et al., 1987, Nucl. Acids Res. 15: 6131-6148), or a chimeric RNA-DNA analog (Inoue et al., 1987, FEBS Lett. 215: 327-330).

In an alternative embodiment, the antisense nucleic acids of the invention are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Such promoters for mammalian cells include, but are not limited to: the SV40 early promoter region (Bemoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded anti-sense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibition of translation of the target RNA can be determined by standard assay techniques.

Therefore, antisense nucleic acids can be routinely designed to target virtually any mRNA sequence, and a cell can be routinely transformed with or exposed to nucleic acids coding for such antisense sequences such that an effective amount of the antisense nucleic acid is expressed. Accordingly the translation of virtually any RNA species in a cell can be inhibited.

Finally, in a further embodiment, RNA aptamers can be introduced into or expressed in a cell. RNA aptamers are specific RNA ligands for proteins, such as for Tat and Rev RNA (Good et al., 1997, Gene Therapy 4: 45-54) that can specifically inhibit their translation.

Methods of Modifying Protein Abundances

Methods of modifying protein abundances include, inter alia, those altering protein degradation rates and those using antibodies (which bind to proteins affecting abundances of activities of native target protein species). Increasing (or decreasing) the degradation rates of a protein species decreases (or increases) the abundance of that species. Methods for controllably increasing the degradation rate of a target protein in response to elevated temperature or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g. 37° C.) and which is hidden to prevent rapid degradation at a lower temperature (e.g., 23° C.) (Dohmen et. al, 1994, Science 263:1273-1276). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art Modish et al., 1995, Molecular Biology of the Cell W. H. Freeman and Co., New York, especially chap 8) with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFRₜₛ are not exposed, ubiquitination of the fusion protein does not occur, degradation is slow, and active target protein levels are high. At higher temperatures (in the absence of methotrexate), lysines internal to Arg-DHFRₜₛ are exposed, ubiquitination of the fusion protein occurs, degradation is rapid, and active target protein levels are low. Heat activation is blocked by exposure methotrexate. This method is adaptable to other N-terminal degrons which are responsive to other inducing factors, such as drugs and temperature changes.

Target protein abundances and also, directly or indirectly, their activities can also be decreased by (neutralizing) antibodies. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g. interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below), and assaying the concentration of the wild-type amount or activities of the target protein by standard means (such as immunoassays) known in the art. The net activity of the wild-type form can be assayed by assay means appropriate to the known activity of the target protein.

Antibodies can be introduced into cells in numerous fashions, including, for example, microinjection of antibodies into a cell (Morgan et al., 1988, Immunology Today 9:84-86) or transforming hybridoma mRNA encoding a desired antibody into a cell (Burke et al., 1984, Cell 36:847-858). In a further technique, recombinant antibodies can be engineering and ectopically expressed in a wide variety of non-lymphoid cell types to bind to target proteins as well as to block target protein activities (Biocca et al, 1995, Trends in Cell Biology 5:248-252). A first step is the selection of a particular monocolonal antibody with appropriate specificity to the target protein (see below). Then sequences encoding the variable regions of the selected antibody can be cloned into various engineered antibody formats, including, for example, whole antibody, Fab fragments, Fv fragments, single chain Fv fragments (V_{H} and V_{L} regions united by a peptide linker) (“ScFv” fragments), diabodies (two associated ScFv fragments with different specificities), and so forth (Hayden et al., 1997, Current Opinion in Immunology 9:210-212). Intracellularly expressed antibodies of the various formats can be targeted into cellular compartments (e.g., the cytoplasm, the nucleus, the mitochondria, etc.) by expressing them as fusions with the various known intracellular leader sequences (Bradbury et al., 1995, Antibody Engineering (vol. 2) (Borrebaeck ed.), pp 295-361, IRL Press). In particular, the ScFv format appears to be particularly suitable for cytoplasmic targeting.

Antibody types include, but are not limited to, polyclonal, monoclonal, chimeric, single chain, Fab fragments, and an Fab expression library. Various procedures known in the art may be used for the production of polyclonal antibodies to a target protein. For production of the antibody, various host animals can be immunized by injection with the target protein, such host animals include, but are not limited to, rabbits, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Calnette-Guerin (BCG) and corynebacterium parvum.

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein (1975, Nature 256: 495-497), the trioma technique, the human B-cell hybridoma technique Kozbor et al., 1983, Immunology Today 4: 72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. USA 80: 2026-2030), or by transforming human B cells with EBV virus in vitro (Cole et al., 1985, in Monoclonal Antibodies and Cancer Therapy, Alan R. Liss, Inc., pp. 77-96). In fact, according to the invention, techniques developed for the production of “chimeric antibodies” (Morrison et al., 1984, Proc. Natl. Acad. Sci. USA 81: 6851-6855; Neuberger et al., 1984, Nature 312:604-608; Takeda et al., 1985, Nature 314: 452-454) by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display (Marks et al., 1992, J. Biol. Chem. 267:16007-16010). Using this technique, libraries of up to 10¹² different antibodies have been expressed on the surface of fd filamentous phage, creating a “single pot” in vitro immune system of antibodies available for the selection of monoclonal antibodies (Griffiths et al., 1994, EMBO J. 13:3245-3260). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single chain antibodies (U.S. Pat. No. 4,946,778) can be adapted to produce single chain antibodies specific to the target protein. An additional embodiment of the invention utilizes the techniques described for the construction of Fab expression libraries (Huse et al., 1989, Science 246: 1275-1281) to allow rapid and easy identification of monoclonal Fab fragments with the desired specificity for the target protein.

Antibody fragments that contain the idiotypes of the target protein can be generated by techniques known in the art. For example, such fragments include, but are not limited to: the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent, and Fv fragments.

In the production of antibodies, screening for the desired antibody can be accomplished by techniques known in the art, e.g., ELISA (enzyme-linked immunosorbent assay). To select antibodies specific to a target protein, one may assay generated hybridomas or a phage display antibody library for an antibody that binds to the target protein.

Methods of Modifying Protein Activities

Methods of directly modifying protein activities include, inter alia, dominant negative mutations, specific drugs (used in the sense of this application), and also the use of antibodies, as previously discussed.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target (Hershkowitz, 1987, Nature 329:219-222). In the case of active monomeric forms, over expression of an inactive form can cause competition for natural substrates or ligands sufficient to significantly reduce net activity of the target protein. Such over expression can be achieved by, for example, associating a promoter of increased activity with the mutant gene. Alternatively, changes to active site residues can be made so that a virtually irreversible association occurs with the target ligand. Such can be achieved with certain tyrosine kinases by careful replacement of active site serine residues (Perlmutter et al., 1996, Current Opinion in Immunology 8:285-290).

In the case of active multimeric forms, several strategies can guide selection of a dominant negative mutant. Multimeric activity can be decreased by expression of genes coding exogenous protein fragments that bind to multimeric association domains and prevent multimer formation. Alternatively, over expression of an inactive protein unit of a particular type can tie up wild-type active units in inactive multimers, and thereby decrease multimeric activity (Nocka et al., 1990, The EMBO J. 9:1805-1813). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities.

In addition to dominant negative mutations, mutant target proteins that are sensitive to temperature (or other exogenous factors) can be found by mutagenesis and screening procedures that are well-known in the art.

Also, one of skill in the art will appreciate that expression of antibodies binding and inhibiting a target protein can be employed as another dominant negative strategy.

Finally, alternatively to techniques involving mutations, activities of certain target proteins can be altered by exposure to exogenous drugs or ligands. In a preferable case, a drug is known that interacts with only one target protein in the cell and alters the activity of only that one target protein. Exposure of a cell to that drug thereby modifies the cell. The alteration can be either a decrease or an increase of activity. Less preferably, a drug is known and used that alters the activity of only a few (e.g., 2-5) target proteins with separate, distinguishable, and non-overlapping effects.

### 5.4 Comparison of the Transcript Profile of an Organism with A Desired Phenotype to the Compendium

In one embodiment, the transcript profile of a cell type or organism having a desired phenotype can be compared to profiles in the compendium in order to infer, by quantitating the degree of similarity between profiles, the genetic cause of the phenotype. In another embodiment, a transcript profile of a cell type or organism having an unknown phenotype can be compared to transcript profiles in the compendium and to transcript profiles of organisms having desired phenotypes to elucidate the likely phenotype of the organism. In another embodiment, a transcript profile of a cell type or organism can be compared to profiles in the compendium in order to determine if a genotype associated with a phenotype of interest is present or absent in the cell type or organism.

The methods of the present invention are also useful for monitoring the results of genetic engineering or selective breeding of, inter alia, livestock and crop plants. In one embodiment, results of genetic engineering or selective breeding attempts can be profiled to determine if the profile matches that expected from the desired modification. In another embodiment, cross-breeding products can be profiled in order to determine whether desirable or undesirable effects are present in addition to the expected effects. In yet another embodiment, immature products of breeding or genetic engineering can be profiled in order to predict mature phenotypic traits (see Section 5.8, infra).

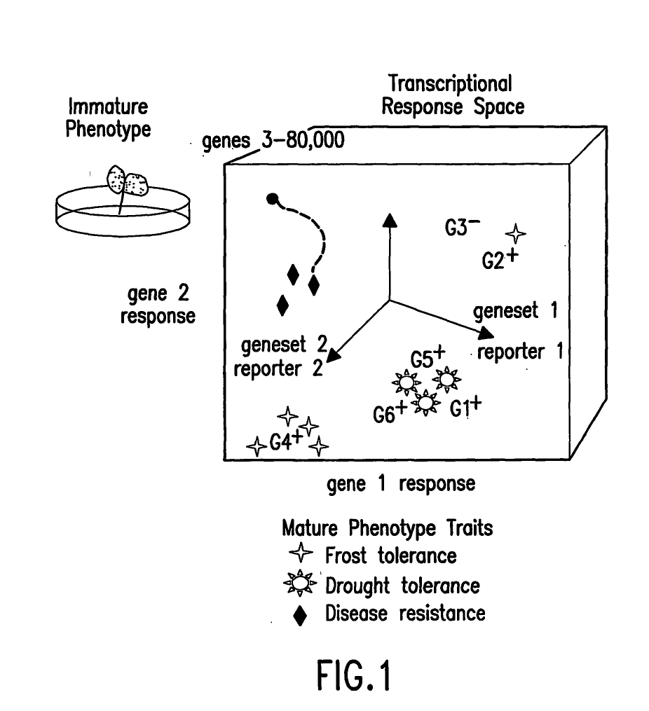

FIG. 1 is an exemplary illustration of transcriptional response space in which there are measurements of phenotypes and genetic landmarks. Each point in the space represents a transcript profile, which is a set of measurements of mRNA abundances or other abundances relative to some baseline condition (e.g., wild-type cells). These measurements cover a plurality of genes expressed in the cell being studied. For example, for a full yeast genome, there would be approximately 6,000 measurements represented by one point in FIG. 1. In this case, transcriptional response space would have 6,000 dimensions.

The genetic landmarks, denoted G1, G2, etc., may be, for example, gene deletion, over-expression or under-expression strains. In FIG. 1, individuals of the frost-resistant phenotype are shown grouped around G4⁺ and G2⁺, which denote over-expression of the G4 gene and the G2 gene, respectively. Thus, some of the individuals having the frost-resistant phenotype have it by virtue of over-expression of G4, while at least one individual has the phenotype by virtue of over-expression of G2. The G4⁺ and G2⁺ landmark profiles are dissimilar, which is shown by the relatively great distance between them. Thus, G4 and G2 are likely to be involved in different biological pathways, and multiple genes associated with the frost-resistant phenotype are indicated. Likewise, several different genetic perturbations may contribute to one particular phenotype because the genes are involved in similar biochemical pathways. In this case, transcript profiles corresponding to each of these perturbations will group closely together in the transcriptional response space of FIG. 1, and all of the genes in the similar biochemical pathways will be implicated by individual phenotypes whose profiles occur nearby. Thus, in the case of the drought resistant phenotype, over-expression of G1, G5 or G6 all produce this phenotype and are clustered together in space because they have similar profiles, indicating the involvement of these genes in similar biochemical pathways.

A mutant is profiled by measuring the same set of mRNA or other abundances that comprise the transcriptional response space of FIG. 1, and the profile is placed therein (filled circle). Similarity between this mutant profile and landmark profiles is measured by proximity of the profiles in the space, which is quantitatively defined.

In one embodiment, the measure of profile similarity is the negative of the Euclidean distance, given by Equation 3:

$$d = {\sum }_{k}^{} {( {x}_{ik} - {x}_{jk} )}^{2} \qquad ( 3 )$$

where k is a gene index that identifies a particular gene, where xᵢₖ, xⱼₖ are the logarithms of the expression ratios between the perturbed and unperturbed (e.g. baseline) conditions for gene k in profiles i and j, respectively. Thus, the more dissimilar the xᵢₖ and xⱼₖ measurements are for each gene k, the greater the absolute value of d, and the greater the distance between profiles i and j in transcriptional response space.

In a preferred embodiment, the similarity between profiles is measured by a weighted correlation coefficient, r, given by Equation 4:

$$r = {\sum }_{k}^{} {x}_{ik} {x}_{jk} / {( {\sum }_{k}^{} {x}_{ik}^{2} {\sum }_{1}^{} {x}_{j1}^{2} )}^{1 / 2} \qquad ( 4 )$$

xᵢₖ is qᵢₖ/aᵢₖ and xⱼₖ is qⱼₖ/σⱼₖ, where qᵢₖ and qⱼₖ are the logarithms of the expression ratios between the perturbed and baseline conditions for gene k in profiles i and j, respectively, and σᵢₖ and σⱼₖ are the expected root mean square uncertainties in the measurements of qᵢₖ and qⱼₖ, respectively. This is an optimally-weighted measure of correlation between two profiles, given the error estimate, σ, on each data point.

Two profiles are “similar” for the purposes of the methods of the present invention if they have a statistically significant correlation. For example, values of r significantly different from zero are those that have a small likelihood of occurring by chance under the hypothesis that the profiles are in fact not correlated Under the uncorrelated hypothesis, the probability distribution of r is approximated by equation 5:

z=(½) [ln(l+r)−ln(1−r)] (5)

wherein z is normally distributed with standard error 1/(n−3)^{1/2} and n is the total number of measurements (Fisher, 1921, Metron 13). Thus, a pair of values r and n that resulted in a z value of greater than 2/(n−3)^{1/2} indicates profile similarity at a two standard deviation level of significance.

A non-parametric approach to assigning a probability to any r value is to randomize the order of the elements in the data vectors (i.e., the gene indices), and then generate a Monte Carlo distribution of r arising from the rearranged data, which satisfies the uncorrelated hypothesis. The value of r computed from the actual data is then compared to this distribution in order to assign a likelihood that the correlation is not random.

Similarity between an individual profile and a genetic landmark profile does not always guarantee that the particular gene that is affected to produce the genetic landmark profile is responsible for the observed phenotype in the organism that produced the individual profile. One complication is that disruption of different genes involved in the same biological pathway may result in very similar transcript profiles, since the same transcriptional signals are disturbed. By “pathway,” as used herein, is meant any chain of molecular events leading to a measurable change in, inter alia, transcription, translation or protein activities, not just classical metabolic pathways. In this case, profile similarity indicates that the phenotype is related to one or more of the genes in the pathway, which narrows the search for the genes that cause the phenotype.

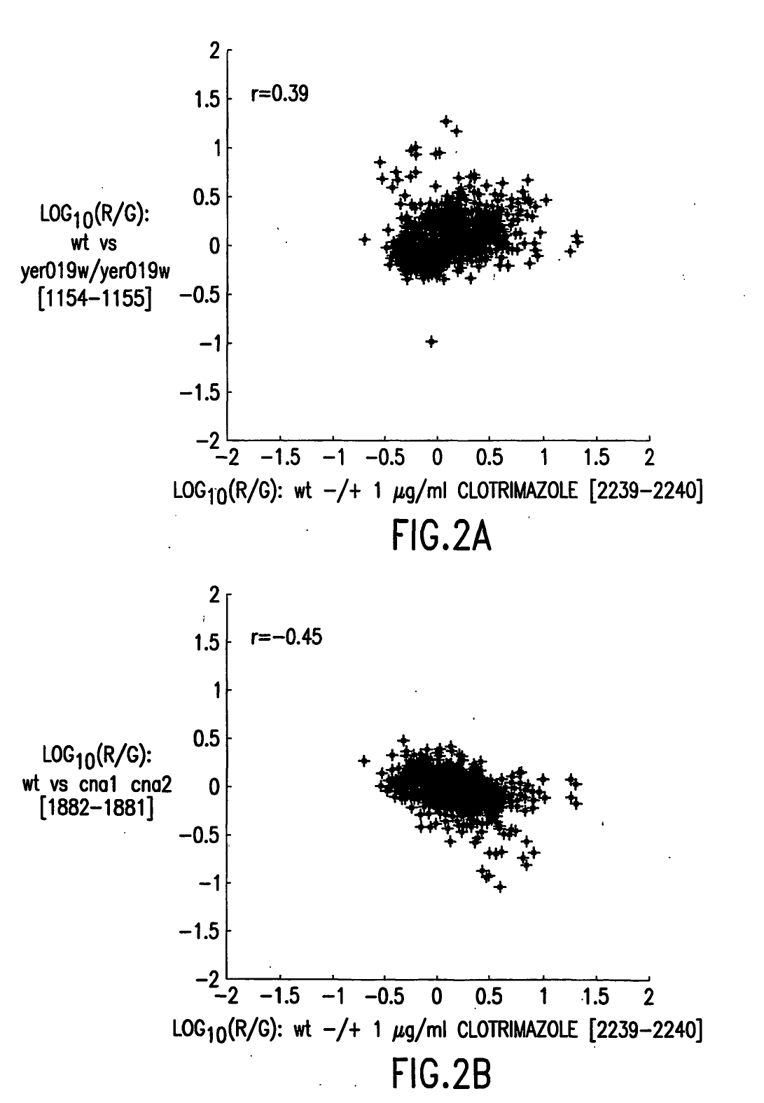

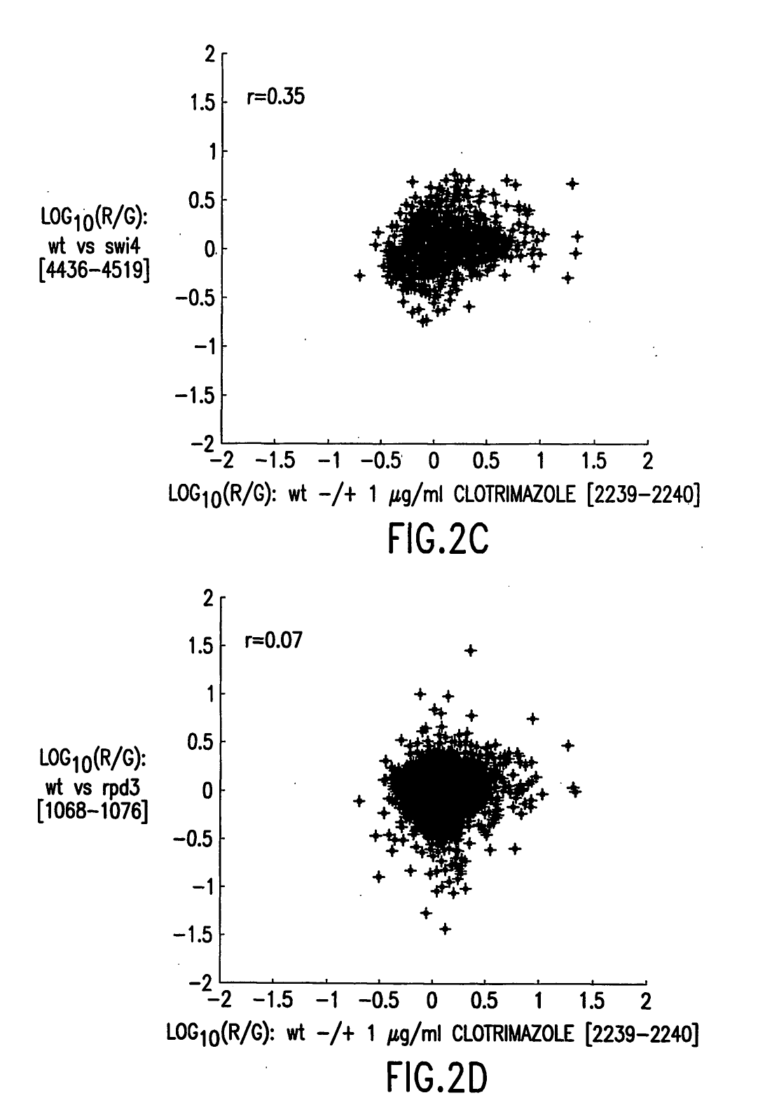

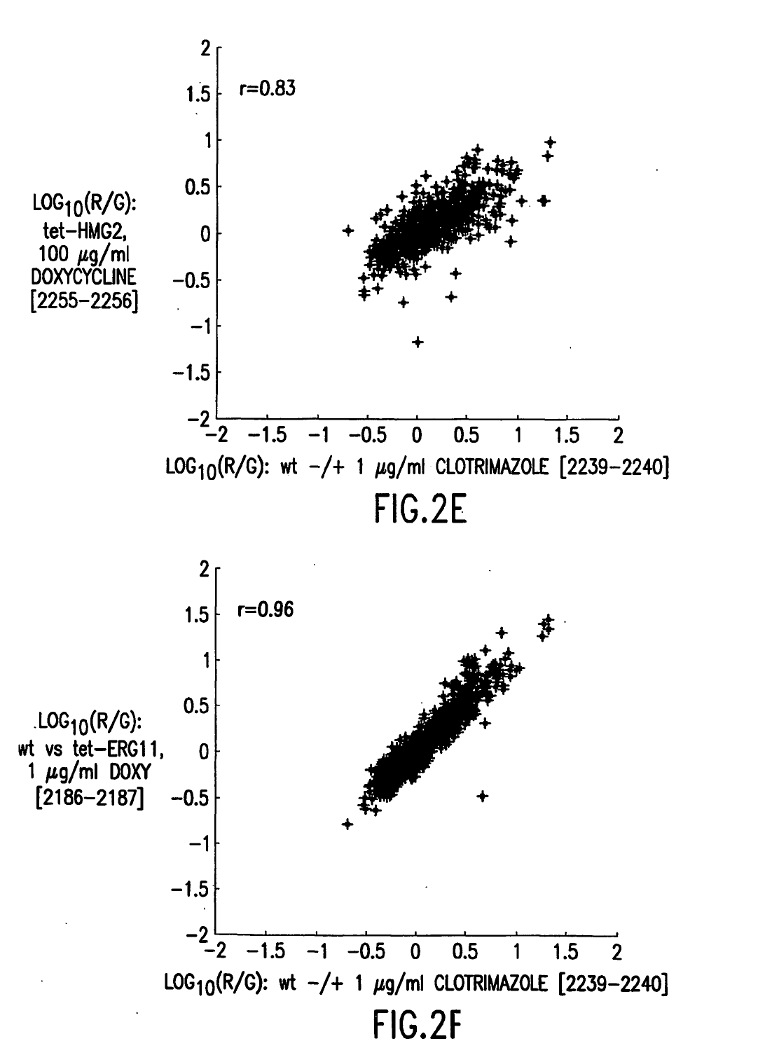

In many cases, the particular gene in the pathway that is responsible for a particular phenotype can be identified. An example of this is shown in FIG. 2, which compares the transcript profile of yeast in which the function or activity of the Erg11 protein is inhibited by the chemical clotrimazole α-axes) with various landmark transcript profiles of mutants, including deletion mutants yer019w/yer019w (FIG. 2a), cna1 cna2 (FIG. 2b), swi4 (FIG. 2c), and rpd3 (FIG. 2d), and perturbations in the HMG2 (FIG. 2e) and ERG11 genes (FIG. 2f) (y-axes). For each profile, the gene transcript abundances were compared to gene transcript abundances of wild-type (wt) yeast. Using the representation of FIG. 2, if the two profiles compared in a graph were identical, then each point in the graph would have x (the abundance of gene k in the clotrimazole profile) equal to y (the abundance of gene k in the landmark profile) and would lie along the diagonal of the graph in a straight line. In this case, the correlation coefficient, r, would be 1. Thus, the degree of linearity of each plot, which is reflected in the value of the correlation coefficient, is indicative of the degree of similarity between the two profiles being compared.

As is evidenced in FIG. 2, inhibition of the function or activity of the Erg 11 protein with clotrimazole causes a transcriptional response which is similar to that of yeast having a disruption of the ERG11 gene (r=0.96) (FIG. 2f) and to that of yeast having a disruption of the HMG2 gene (r=0.83) (FIG. 2e). Although both ERG11 and HMG2 are genes involved in the ergosterol biosynthesis pathway, the clotrimazole profile is clearly more similar to the disruption of ERG11, demonstrating the ability of the methods of the present invention to distinguish the actual causative gene from other genes in the same pathway.

### 5.5 The Use of Genesets to Reduce the Dimensionality of Transcriptional Response Space

In one embodiment of the present invention, the number of genes to be monitored in a given profile is reduced by monitoring sets of co-varying genes (genesets), or biological pathway reporters, instead of individual genes. This not only reduces the dimensionality of transcriptional response space (as depicted by the labeled axes in FIG. 1), but also increases the robustness of the measurements. Methods for classifying genes into genesets and for using them in profile analysis are disclosed in co-pending U.S. patent application Ser. Nos. 09/179,569 (filed Oct. 27, 1998), 09/220,275 (filed Dec. 23, 1998), PCT International Publication WO 00/24936, published May 4, 2000, and Ser. No. 09/428,427 (filed Oct. 27, 1999), which are incorporated herein by reference in their entireties.

Certain genes tend to increase or decrease their expression in groups, as shown in FIG. 2 by portions of columns that have the same shade of gray. For example, the set of genes around column 425 labeled “Mitochondrial Function” are all co-regulated, as are the genes involved in mating (labeled “Mating;” approximately columns 480-510). Genes tend to increase or decrease their rates of transcription together when they possess similar regulatory sequence patterns, i.e., transcription factor binding sites. This is the mechanism for coordinated response to particular signaling inputs (see, e.g., Madhani and Fink, 1998, The riddle of MAP kinase signaling specificity, Transactions in Genetics 14:151-155; Arnone and Davidson, 1997, The hardwiring of development: organization and function of genomic regulatory systems, Development 124:1851-1864). Separate genes which make different components of a necessary protein or cellular structure will tend to co-vary. Duplicated genes (see, e.g., Wagner, 1996, Genetic redundancy caused by gene duplications and its evolution in networks of transcriptional regulators, Biol. Cybern. 74:557-567) will also tend to co-vary to the extent mutations have not led to functional divergence in the regulatory regions. Further, because regulatory sequences are modular (see, e.g., Yuh et al.,1998, Genomic cis-regulatory logic: experimental and computational analysis of a sea urchin gene, Science 279:1896-1902), the more modules two genes have in common, the greater the variety of conditions under which they are expected to co-vary their transcriptional rates. Separation between modules is also an important determinant since co-activators also are involved. In summary therefore, for any genetic mutation, it is expected that genes will not all vary independently, and that there are simplifying subsets of genes and proteins that will co-vary. These co-varying sets of genes form a complete basis in the mathematical sense with which to describe all the profile changes arising from a particular genetic mutation.

### 5.5.1 Determining Co-Varying Sets

The methods of the present invention involve arranging or grouping cellular constituents in the response profiles according to their tendency to co-vary in response to a perturbation. In particular, this Section describes specific embodiments for arranging the cellular constituents into co-varying sets.

Clustering Algorithms:

Preferably, the basis or co-varying sets of the present invention are identified by means of a clustering algorithm (i.e., by means of “clustering analysis”). Clustering algorithms of this invention may be generally classified as “model-based” or “model-independent” algorithms. In particular, model-based clustering methods assume that co-varying sets or clusters map to some predefined distribution shape in the cellular constituent “vector space.” For example, many model-based clustering algorithms assume ellipsoidal cluster distributions having a particular eccentricity. By contrast, model-independent clustering algorithms make no assumptions about cluster shape. As is recognized by those skilled in the art, such model-independent methods are substantially identical to assuming “hyperspherical” cluster distributions. Hyperspherical cluster distributions are generally preferred in the methods of this invention, e.g., when the perturbation vector elements vₗ^{(m)} have similar scales and meanings, such as the abundances of different mRNA species.

The clustering methods and algorithms of the present invention may be further classified as “hierarchical” or “fixed-number of groups” algorithms (see, e.g. S-Plus Guide to Statistical and Mathematical Analysis v.3.3, 1995, MathSoft, Inc.: StatSci. Division, Seattle, Wash.). Such algorithms are well known in the art (see, e.g., Fukunaga, 1990, Statistical Pattern Recognition, 2nd Ed., San Diego: Academic Press; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, New York: Academic Press), and include, e.g., hierarchical agglomerative clustering algorithms, the “k-means” algorithm of Hartigan (supra), and model-based clustering algorithms such as mclust by MathSoft, Inc. Preferably, hierarchical clustering methods and/or algorithms are employed in the methods of this invention. In a particularly preferred embodiment, the clustering analysis of the present invention is done using the hclust routine or algorithm (see, e.g., ‘hclust’ routine from the software package S-Plus, MathSoft, Inc., Cambridge, Mass.).

The clustering algorithms used in the present invention operate on a table of data containing measurements of a plurality of cellular constituents, preferably gene expression measurements, such as those described in Section ______ above. Specifically, the data table analyzed by the clustering methods of the present invention comprise an N×K array or matrix wherein N is the total number of conditions or perturbations and K is the number of cellular constituents measured or analyzed.

The clustering algorithms of the present invention analyze such arrays or matrices to determine dissimilarities between cellular constituents. Mathematically, dissimilarities between cellular constituents i and j are expressed as “distances” I_{i,j}. For example, in one embodiment, the Euclidian distance is determined according to the Equation 6:

$${I}_{i , j} = {( {\sum }_{m}^{} {\vert {v}_{i}^{( m )} - {v}_{j}^{( m )} \vert}^{2} )}^{1 / 2} \qquad ( 6 )$$

In Equation 6, above, vᵢ^{(m)}and vⱼ^{(m)} are the responses of cellular constituent i and j, respectively, to the perturbation m. In other embodiments, the Euclidian distance in Equation 6, above, is squared to place progressively greater weight on cellular constituents that are further apart. In alternative embodiments, the distance measure Iᵢⱼ is the Manhattan distance provided by Equation 7:

$${I}_{i , j} = {\sum }_{m}^{} \vert {v}_{i}^{( m )} - {v}_{j}^{( m )} \vert \qquad ( 7 )$$

In embodiments wherein the response profile data is categorical (e.g. wherein each element vᵢ^{(m)}=1 or 0), the distance measure is preferably a percent disagreement defined by Equation 8:

$${I}_{i , j} = \frac{No . of {v}_{i}^{( m )} \neq {v}_{j}^{( m )}}{N} \qquad ( 8 )$$

In a particularly preferred embodiment, the distance is defined as I_{i,j}=1−r_{i,j}, where r_{i,j} is the “correlation coefficient” or normalized “dot product” between the response vectors vᵢ and vⱼ. In particular, r_{i,j} is defined by Equation 9, below:

$${r}_{i , j} = \frac{{v}_{i} \cdot {v}_{j}}{\vert {v}_{i} ‖ {v}_{j} \vert} \qquad ( 9 )$$

In Equation 9, the dot product vᵢ·vⱼ is defined according to Equation 10:

$${v}_{i} \cdot {v}_{j} = {\sum }_{m}^{} ( {v}_{i}^{( m )} \times {v}_{j}^{( m )} ) \qquad ( 10 )$$

Further, the quantities |vᵢ| and |vⱼ| in Equation 9 are provided by the relations |vᵢ| (vᵢ·vⱼ)^{1/2}, and |vⱼ|=(vⱼ·vⱼ)^{1/2}.

In still other embodiments, the distance measure can some other distance measure known in the art, such as the Chebychev distance, the power distance, and percent disagreement, to name a few. Most preferably, the distance measure is appropriate to the biological questions being asked, e.g., for identifying co-varying and/or co-regulated cellular constituents including co-varying or co-regulated genes. For example, in a particularly preferred embodiment, the distance measure I_{i,j}=1−r_{i,j} with the correlation coefficient which comprises a weighted dot product of the response vectors vᵢ and vⱼ. Specifically, in this preferred embodiment, r_{i,j} is preferably defined by Equation 11:

$${r}_{i , j} = \frac{{\sum }_{m}^{} \frac{{v}_{i}^{( m )} {v}_{j}^{( m )}}{{σ}_{i}^{( m )} {σ}_{i}^{( m )}}}{{[ {\sum }_{m}^{} {( \frac{{v}_{i}^{( m )}}{{σ}_{i}^{( m )}} )}^{2} {\sum }_{m}^{} {( \frac{{v}_{j}^{( m )}}{{σ}_{j}^{( m )}} )}^{2} ]}^{1 / 2}} \qquad ( 11 )$$

In Equation 11, above, the quantities σᵢ^{(m)} and σⱼ^{(m)} are the standard errors associated with the measurement of the i'th and j'th cellular constituents, respectively, in experiment m.

The correlation coefficients provided by Equations 9 and 11 are bounded between values of +1, which indicates that the two response vectors are perfectly correlated and essentially identical, and −1, which indicates that the two response vectors are “anti-correlated” or “anti-sense” (i.e., are opposites). These correlation coefficients are particularly preferably in embodiments of the invention where cellular constituent sets or clusters are sought of constituents which have responses of the same sign. However, in other embodiments, it can be preferable to identify cellular constituent sets or clusters which are co-regulated or involved in the same biological responses or pathways but comprise both similar and anti-correlated responses. In such embodiments, it is preferable to use the absolute value of the correlation coefficient provided by Equation 9 or 11; i.e., |r_{i,j}| as the correlation coefficient.

In still other embodiments, the relationships between co-regulated and/or co-varying cellular constituents may be even more complex, such as in instances wherein multiple biological pathways (for example, multiple signaling pathways) converge on the same cellular constituent to produce different outcomes. In such embodiments, it is preferable to use a correlation coefficient r_{i,j}=r_{i,j}^{(change)} which is capable of identifying co-varying and/or co-regulated cellular constituents irrespective of the sign. The correlation coefficient specified by Equation 12, below, is particular useful in such embodiments.

$${r}_{i , j}^{( change )} = \frac{{\sum }_{m}^{} \vert \frac{{v}_{i}^{( m )}}{{σ}_{i}^{( m )}} \vert \vert \frac{{v}_{j}^{( m )}}{{σ}_{j}^{( m )}} \vert}{{[ {\sum }_{m}^{} {( \frac{{v}_{i}^{( m )}}{{σ}_{i}^{( m )}} )}^{2} {\sum }_{m}^{} {( \frac{{v}_{j}^{( m )}}{{σ}_{j}^{( m )}} )}^{2} ]}^{1 / 2}} \qquad ( 12 )$$

Generally, the clustering algorithms used in the methods of the invention also use one or more linkage rules to group cellular constituents into one or more sets or “clusters.” For example, single linkage or the nearest neighbor method determines the distance between the two closest objects (i.e., between the two closest cellular constituents) in a data table. By contrast, complete linkage methods determine the greatest distance between any two objects (i.e., cellular constituents) in different clusters or sets. Alternatively, the unweighted pair-group average evaluates the “distance” between two clusters or sets by determining the average distance between all pairs of objects (i.e., cellular constituents) in the two clusters. Alternatively, the weighted pair-group average evaluates the distance between two clusters or sets by determining the weighted average distance between all pairs of objects in the two clusters, wherein the weighing factor is proportional to the size of the respective clusters. Other linkage rules, such as the unweighted and weighted pair-group centroid and Ward's method, are also useful for certain embodiments of the present invention (see, e.g., Ward, 1963, J. Am. Stat. Assn 58:236; Hartigan, 1975, Clustering Algorithms, New York: Wiley).

In particularly preferred embodiments, an agglomerative hierarchical clustering algorithm is used. Such algorithms are known in the art and described, e.g., in Hartigan, supra. Briefly, the algorithm preferably starts with each object (e.g., each cellular constituent) as a separate group. In each successive step, the algorithm identified the two most similar objects by finding the minimum of all the pair-wise similarity measures, merges them into one object (i.e., into one “cluster”) and updates the between-cluster similarity measures accordingly. The procedure continues until all objects are found in a single group. When merging two closest objects, a heuristic criterion of average linkage is preferably employed to redefine the between-cluster similarity measures. Since two objects are combined at each similarity level, such a clustering algorithm clustering yields a rigid hierarchical structure among objects and defines their memberships.

Once a clustering algorithm has grouped the cellular constituents from the data table into sets or clusters, e.g., by application of linkage rules such as those described supra, a clustering “tree” may be generated to illustrate the clusters of cellular constituents so determined. Genesets may be readily defined based on the branchings of a clustering tree. In particular, genesets may be defined based on the many smaller branchings of a clustering tree, or, optionally, larger genesets may be defined corresponding to the larger branches of a clustering tree. Preferably, the choice of branching level at which genesets are defined matches the number of distinct response pathways expected. In embodiments wherein little or no information is available to indicate the number of pathways, the genesets should be defined according to the branching level wherein the branches of the clustering tree are “truly distinct.”

“Truly distinct,” as used herein, may be defined, e.g., by a minimum distance value between the individual branches. Typically, the distance values between truly distinct genesets are in the range of 0.2 to 0.4, where a distance of zero corresponds to perfect correlation and a distance of unity corresponds to no correlation. However, distances between truly distinct genesets may be larger in certain embodiments, e.g., wherein there is poorer quality data or fewer experiments n in the response profile data. Alternatively, in other embodiments, e.g., having better quality data or more experiments n in the profile dataset, the distance between truly distinct genesets may be less than 0.2.

Statistical Significance:

Preferably, truly distinct cellular constituent sets are defined by means of an objective test of statistical significance for each bifurcation in the clustering tree. For example, in one aspect of the invention, truly distinct cellular constituent sets are defined by means of a statistical test which uses Monte Carlo randomization of the experiment index m for the responses of each cellular constituent across the set of experiments. For example, in one preferred embodiment, the experiment index m of each cellular constituent's response vᵢ^{(m)} is randomly permutated, as indicated by Equation 13:

vᵢ^{(m)→v}ᵢ^{π(m)} (13)

More specifically, a large number of permutations of the experiment index m is generated for each cellular constituent's response. Preferably, the number of permutations is from 50 to about 1000, more preferably from 50 to about 100. For each branching of the original clustering tree, and for each permutation of the experiment index:

(1) hierarchical clustering is performed on the permutated data, preferably using the same clustering algorithm as used for the original unpermuted data; and

(2) the fractional improvement f in the total scatter is computed with respect to the cluster centers in going from one cluster to two clusters.

In particular, the fractional improvement f is computed according to Equation 14, below:

$$f = 1 - \frac{\sum {D}_{i}^{( 1 )}}{\sum {D}_{i}^{( 2 )}} \qquad ( 14 )$$

In Equation 14, Dₗ is the square of the distance measure for cellular constituent i with respect to the center (i.e., the mean) of its assigned cluster. The superscripts (1) and (2) indicate whether the square of the distance measure Dᵢ is made with respect to (1) the center of its entire branch, or (2) the center of the appropriate cluster out of the two clusters. The distance function Dᵢ in Equation 14 may be defined according to any one of several embodiments. In particular, the various embodiments described supra for the definition of I_{i,j} may also be used to define Dᵢ in Equation 14.

The distribution of fractional improvements obtained from the above-described Monte Carlo methods provides an estimate of the distribution under the null hypothesis, i.e., the hypothesis that a particular branching in a cluster tree is not significant or distinct. A significance can thus be assigned to the actual fractional improvement (i.e., the fraction improvement of the unpermuted data) by comparing the actual fractional improvement to the distribution of fractional improvements for the permuted data. Preferably, the significance is expressed in terms of the standard deviation of the null hypothesis distribution, e.g., by fitting a log normal model to the null hypothesis distribution obtained from the permuted data. Numbers greater than about 2, for example, indicate that the branching is significant at the 95% confidence level.

In more detail, an objective statistical test is preferably employed to determine the statistical reliability of the grouping decisions of any clustering method or algorithm. Preferably, a similar test is used for both hierarchical and non-hierarchical clustering methods. More preferably, the statistical test employed comprises (a) obtaining a measure of the compactness of the clusters determined by one of the clustering methods of this invention, and (b) comparing the obtained measure of compactness to a hypothetical measure of compactness of cellular constituents regrouped in an increased number of clusters. For example, in embodiments wherein hierarchical clustering algorithms, such as hclust, are employed, such a hypothetical measure of compactness preferably comprises the measure of compactness for clusters selected at the next lowest branch in a clustering tree. Alternatively, in embodiments wherein non-hierarchical clustering methods or algorithms are employed, e.g., to generate N clusters, the hypothetical measure of compactness is preferably the compactness obtained for N+1 clusters by the same methods.

Cluster compactness may be quantitatively defined, e.g., as the mean squared distance of elements of the cluster from the “cluster mean,” or, more preferably, as the inverse of the mean squared distance of elements from the cluster mean. The cluster mean of a particular cluster is generally defined as the mean of the response vectors of all elements in the cluster. However, in certain embodiments, e.g., wherein the absolute value of Equation 9 or 11 is used to evaluate the distance metric (i.e., Iᵢⱼ=1−|rᵢⱼ|) of the clustering algorithm, such a definition of cluster mean is problematic. More generally, the above definition of mean is problematic in embodiments wherein response vectors can be in opposite directions such that the above defined cluster mean could be zero. Accordingly, in such embodiments, it is preferable to choose a different definition of cluster compactness such as, but not limited to, the mean squared distance between all pairs of elements in the cluster. Alternatively, the cluster compactness may be defined to comprise the average distance (or more preferably the inverse of the average distance) from each element (e.g., cellular constituent) of the cluster to all other elements in that cluster.

Preferably, step (b) above of comparing cluster compactness to a hypothetical compactness comprises generating a non-parametric statistical distribution for the changed compactness in an increased number of clusters. More preferably, such a distribution is generated using a model which mimics the actual data but has no intrinsic clustered structures (i.e., a “null hypothesis” model). For example, such distributions may be generated by (a) randomizing the perturbation experiment index m for each actual perturbation vector vᵢ^{(m)}, and (b) calculating the change in compactness which occurs for each distribution, e.g., by increasing the number of clusters from N to N+1 (non-hierarchical clustering methods), or by increasing the branching level at which clusters are defined (hierarchical methods).

In an exemplary embodiment, the increased compactness is given by the parameter E, which is defined by Equation 15, below:

$$E = \frac{{I}_{mean}^{( N )} - {I}_{mean}^{( N + 1 )}}{{I}_{mean}^{( N + 1 )}} \qquad ( 15 )$$

However, other definitions that are apparent to those skilled in the art can also be used in the statistical methods of this invention. In general, the exact definition of E is not crucial provided it is monotonically related to increase in cluster compactness.

The statistical methods of this invention provide methods to analyze the significance of E. Specifically, these methods provide an empirical distribution approach for the analysis of E by comparing the actual increase in compactness, E₀, for actual experimental data to an empirical distribution of E values determined from randomly permuted data (e.g., by Equation 13 above). Such a translation may comprise, first, randomly swapping the perturbation indices m=1,2 in each perturbation vector with equal probability. More specifically, the coordinates (i.e., the indices) of the vectors in each cluster being subdivided are “reflected” about the cluster center, e.g., by first translating the coordinate axes to the cluster center. Second, the randomly permuted data are re-evaluated by the cluster algorithms of the invention, most preferably by the same cluster algorithm used to determine the original cluster(s), so that new clusters are determined for the permutated data, and a value of E is evaluated for these new clusters (i.e., for splitting one or more of the new clusters). Steps one and two above are repeated for some number of Monte Carlo trials to generate a distribution of E values. Preferably, the number of Monte Carlo trials is from about 50 to about 1000, and more preferably from about 50 to about 100. Finally, the actual increase in compactness, i.e., E₀, is compared to this empirical distribution of E values. For example, if M Monte Carlo simulations are performed, of which x have E values greater than E₀, then the confidence level in the number of clusters may be evaluated from 1−x/M. In particular, if M=100, and x=4, then the confidence level that there is no real significance in increasing the number of clusters is 1−4/100=96%.

The above methods are equally applicable to embodiments comprising hierarchical clusters and/or a plurality of elements (e.g., more than two cellular constituents).

Classification Based Upon Mechanisms of Regulation:

Cellular constituent sets can also be defined based upon the mechanism of the regulation of cellular constituents. For example, genesets can often be defined based upon the regulation mechanism of individual genes. Genes whose regulatory regions have the same transcription factor binding sites are more likely to be co-regulated, and, as such, are more likely to co-vary. In some preferred embodiments, the regulatory regions of the genes of interest are compared using multiple alignment analysis to decipher possible shared transcription factor binding sites (see, e.g., Stormo and Hartzell, 1989, Proc. Natl. Acad. Sci. 86:1183-1187; and Hertz and Stormo, 1995, Proc. of 3rd Intl. Conf. on Bioinformatics and Genome Research, Lim and Cantor, eds., Singapore: World Scientific Publishing Co., Ltd., pp.201-216). For example, the common promoter sequence responsive to Gcn4 in 20 genes is likely to be responsible for those 20 genes co-varying over a wide variety of perturbations.

Co-regulated and/or co-varying genes may also be in the up- or down-stream relationship where the products of up-stream genes regulate the activity of down-stream genes. For example, as is well known to those of skill in the art, there are numerous varieties of gene regulation networks. Accordingly, the methods of the present invention are not limited to any particular kind of gene regulation mechanism. If it can be derived or determined from their mechanisms of regulation, whatever that mechanism happens to be, that two or more genes are co-regulated in terms of their activity change in response to perturbation, those two or more genes may be clustered into a geneset.

In many embodiments of the present invention, knowledge of the exact regulation mechanisms of certain cellular constituents may be limited and/or incomplete. In such embodiments, it may be preferred to combine cluster analysis methods, described above, with knowledge of regulatory mechanisms to derive better defined, i.e., refined cellular constituent sets. For example, in some embodiments, clustering may be used to cluster genesets when the regulation of genes of interest is partially known. In particular, in many embodiments, the number of genesets may be predetermined by understanding (which may be incomplete or limited) or the regulation mechanism or mechanisms. In such embodiments, the clustering methods may be constrained to produce the predetermined number of clusters. For example, in a particular embodiment promoter sequence comparison may indicate that the measured genes should fall into three distinct genesets. The clustering methods described above may then be constrained to generate exactly three genesets with the greatest possible distinction between those three sets.

Refinement of Cellular Constituent Sets:

Cellular constituent sets, such as cellular constituent sets identified by any of the above methods or combinations thereof, may be refined using any of several sources of corroborating information. Examples of corroborating information which may be used to refine cellular constituent sets include, but are by no means limited to, searches for common regulatory sequence patterns, literature evidence for co-regulations, sequence homology (e.g., of genes or proteins), and known shared function.

In preferred embodiments, a cellular constituent database or “compendium” is used for the refinement of genesets. In particularly preferred embodiments the compendium is a “dynamic database.” For example, in certain embodiments, a compendium containing raw data for cluster analysis of cellular constituent sets (e.g., for genesets) is used to continuously update geneset definitions. Such compendia are discussed, in detail, in Section 5.#, below.

Definition of Basis Vectors:

Once cellular constituent sets have been obtained or provided, e.g., by means of a clustering analysis algorithm such as hclst, a set of basis vectors e can be, optionally, obtained or provided based on those cellular constituent sets. Such basis vectors can be used, e.g., for profile projection methods described in Section 5.#, below.

Preferably, the set of basis vectors has K×N dimensions, where K is the number of cellular constituents and N is the number of cellular constituent sets. In particular, the set of basis vectors e obtained or provided from the cellular constituent sets comprises a matrix of basis vectors which can be represented according to Equation 16:

e=[r⁽¹⁾, . . . , e^{(q)}, . . . , e^{(N)}] (16)

Each basis vector, e^{(q)}, in equation 16 can in turn be represented as a column vector according to Equation 17:

$${e}^{( q )} = \begin{matrix}{e}_{1}^{( q )} \\ ⋮ \\ {e}_{i}^{( q )} \\ ⋮ \\ {e}_{K}^{( q )}\end{matrix} \qquad ( 17 )$$

Preferably, the elements eᵢ^{(q)} of the basis vectors are assigned values:

eᵢ^{(q)}=±1, if cellular constituent i is a member of cellular constituent set (i.e., the cluster) q (the sign is preferably chosen so that constituents which are anti-correlated in their responses across a set of perturbations have opposite signs and constituents with positive correlation have the same sign); and

eᵢ^{(q)}=0, if cellular constituent i is not a member of cellular constituent set q.

Alternatively, the non-zero elements of e^{(q)} can be given magnitudes which are proportional to the typical response magnitude of that element in the cellular constituent set q.

In preferred embodiments, the elements eᵢ^{(q)} are normalized so that each e^{(q)} has a length equal to unity, e.g., by dividing each element by the square root of the number of cellular constituents in cellular constituent set q (ie., by the number of elements eᵢ^{(q)} that are non-zero for a particular cellular constituent set index q). In such embodiments, random measurement errors in profiles project onto the basis vectors in such a way that the amplitudes tend to be comparable for each cellular constituent set. Thus, normalization prevents large cellular constituent sets from dominating the results of calculations involving those sets.

Re-Ordering the Cellular Constituent Index:

As noted above, in preferred embodiments of the present invention the cellular constituents are re-ordered according the cellular constituent sets or clusters obtained or provided by the above-described methods and visually displayed. Analytically, such a reordering corresponds to transforming a particular original biological response profile, such as a particular perturbation response profile, e.g., v⁽ⁿ⁾={vᵢ⁽ⁿ⁾} to the re-ordered profile {v_{π(i)}⁽ⁿ⁾}, where i is the cellular constituent index.

### 5.5.2 Grouping Measured Response Profiles

A second aspect of the analytical methods of the present invention involves methods for grouping or clustering and re-ordering of the perturbation response profiles v^{(m)} into clusters or sets which are associated with similar biological effects of a perturbation. Such methods are exactly analogous to the methods described in Section 5.5.1 above. In particular, the methods and operations described in Section 5.5.1 above which are applied to the cellular constituent index i of the perturbation response profile elements vᵢ^{(m)} may also be applied to the perturbation index m.

The result is a visual display in which experiments with similar profiles are place contiguously. Such a display greatly facilitates the identification of co-regulated genesets. In particular, by visually inspecting such a display, a user can readily identify those genesets which co-vary in groups of experiments. Such a display also facilitates the identification of experiments (e.g., particular perturbations such as particular mutations) which are associated with similar biological responses.

The analytical methods of this invention thus include methods of “two-dimensional” cluster analysis. Such two-dimensional cluster analysis methods simply comprise (1) clustering cellular constituents into sets that are co-varying in biological profiles, and (2) clustering biological profiles into sets that effect similar cellular constituents (preferably in similar ways). The two clustering steps may be performed in any order and according to the methods described above.

Such two-dimensional clustering techniques are useful, as noted above, for identifying sets of genes and experiments of particular interest. For example, the two-dimensional clustering techniques of this invention can be used to identify sets of cellular constituents and/or experiments that are associated with a particular biological effect of interest, such as a drug effect. The two-dimensional clustering techniques of this invention can also be used, e.g., to identify sets of cellular constituents and/or experiments that are associated with a particular biological pathway of interest. In one preferred embodiment of the invention, such sets of cellular constituents and/or experiments are used to determine consensus profiles for a particular biological response of interest. In other embodiments, identification of such sets of cellular constituents and/or experiments provide more precise indications of groupings cellular constituents, such as identification of genes involved in a particular biological pathway or response of interest. Accordingly, another preferred embodiment of the present invention provides methods for identifying cellular constituents, particularly new genes, that are involved in a particular biological effect, of interest e.g. a particular biological pathway. Such cellular constituents are identified according to the cluster-analysis methods described above. Such cellular constituents (e.g. genes) may be previously unknown cellular constituents, or known cellular constituents that were not previously known to be associated with the biological effect of interest.

The present invention further provides methods for the iterative refinement of cellular constituent sets and/or clusters of response profiles (such as consensus profiles). In particular, dominant features in each set of cellular constituents and/or profiles identified by the cluster analysis methods of this invention can be “blanked out”, e.g., by setting their elements to zero or to the mean data value of the set. The blanking out of dominant features may done by a user, e.g., by manually selecting features to blank out, or automatically, e.g., by automatically blanking out those elements whose response amplitudes are above a selected threshold. The cluster analysis methods of the invention are then reapplied to the cellular constituent and/or profile data Such iterative refinement methods can be used, e.g., to identify other potentially interesting but more subtle cellular constituent and/or experiment associations that were not identified because of the dominant features.

### 5.5.3 Projecting Onto Basis Cellular Constituent Sets

In another, optional, aspect of the analytical methods of this invention, biological response profiles, including, e.g., perturbation response profiles, can be represented in terms of basis cellular constituent sets. Such methods are commonly known to those skilled in the art as “projection.”

In particular, as noted in Section 5.5.1, above, the basis vectors obtained from a set of cellular constituents, such as from a geneset, can be represented according to a matrix such as the matrix depicted in Equation 18:

e=[e⁽¹⁾, . . . , e^{(q)}, . . . , e^{(N)}] (18)

where basis vector, e^{(q)}, in equation 16 can in turn be represented as a column vector according to Equation 19:

$${e}^{( q )} = \begin{matrix}{e}_{1}^{( q )} \\ ⋮ \\ {e}_{i}^{( q )} \\ ⋮ \\ {e}_{K}^{( q )}\end{matrix} \qquad ( 19 )$$

Likewise, a biological response profile, denoted here asp, can also be represented as a vector of response values for individual cellular constituents, as depicted in Equation 20:

p=[p₁, . . . , pᵢ, . . . , p_{K}] (20)

For example, the biological response profile can be a particular perturbation response profile, v^{(m)} from a compendium of perturbation response profiles. Alternatively, the biological response profile can also be a new response profile, e.g., for a novel experiment. According to the methods of the invention, the response profile p can be optionally represented in terms of the basis vectors as a “projected profile” P by means of the operation given in Equation 21, below:

P=p·e (21)

Equation 21, above, is well known to those skilled in the art as the “matrix dot product” of p and e. As is also recognized by those skilled in the art, the matrix dot product of p and e generates a new vector, represented by Equation 22:

P=[P₁, . . . , P_{q}, . . . , P_{N}] (22)

In particular, each of the elements, P_{q}, of the vector P in Equations 21 and 22 is provided according to Equation 23:

$$P = p \cdot {e}^{( q )} = {\sum }_{i}^{} {p}_{i} \times {e}_{i}^{( q )} \qquad ( 23 )$$

In other embodiments, the projection of a response profile p onto a basis set of cellular constituents simply comprises the average of the expression value (in p) of the genes within each geneset. In some aspects of such embodiments, the average may be weighted, e.g., so that highly expressed genes do not dominate the average value.

Similarities and differences between two or more projected profiles, for example, between P^{(a)} and P^{(b)} are typically more apparent than are similarities between the original profiles, e.g., p^{(a)} and p^{(b)}, before projection. Thus it is often preferable, in practicing the methods of the present invention, to compare projected response profiles. In particular, measurement errors in extraneous genes are typically excluded or averaged out by projection. Thus, any element of a projected profile, e.g., P^{(a)} or P^{(b)}, is less sensitive to measurement error than is the response of a single cellular constituent (i.e., of a single element of the corresponding unprojected response profile p^{(a)} or p^{(b)}). Accordingly, the elements of a projected profile will generally show significant up- or down-regulation at lower levels of perturbation than will the individual elements (i.e., the individual cellular constituents) of the corresponding unprojected response.

Further, as is well known to those skilled in the art, averaging makes a tremendous difference, e.g., in the probabilities of detecting actual events rather than false alarms (see, e.g., Van Trees, H. L., 1968, Detection, Estimation, and Modulation Theory Vol. I, Wiley & Sons). Accordingly, the elements of a projected profile generally also give more accurate (i.e., small fractional error) measures of the amplitude of response at any level of perturbation. Specifically, in most embodiments of the invention there are independent measurement error in the data for each cellular constituent, or such independent errors may be reasonably assumed. In such embodiments, the fractional standard error of the q'th projected profile elements (i.e., of P_{q}) is approximately M_{q}^{-1/2} times the average fractional error of the individual cellular constituents, where M_{q} is the number of cellular constituents in the q'th cellular constituent sets. Accordingly, if the average measured up or down regulation of an individual cellular constituent is significant at x standard deviations, the projected profile element will be significant at M_{q}^{1/2}x standard deviations.

Finally, because they are derived from observations of co-variance and/or co-regulation, the basis cellular constituents can frequently be directly associated with the biology, e.g., with the biological pathways, of the individual response profile. Thus, the basis cellular constituents function as matched detectors for their individual response components.

### 5.5.4 Consensus Profiles

In a specific embodiment of the invention, one or more consensus profiles is determined for a set of perturbation response profiles, such as in a database or “compendium” of perturbation response profiles. The present invention provides analytical methods that can be used to compare particular biological response profiles (e.g., particular perturbation response profiles such as perturbation response profiles from particular mutations) of interest to such consensus profiles.

Determining Consensus Profiles:

In preferred embodiments, the consensus profiles P^{(C)} of the invention are defined as the intersection of the sets of cellular constituents activated (or de-activated) by members of a group of experimental conditions, such as a group of perturbations (e.g., a group of particular mutations). Such intersections can be identified by either qualitative or quantitative methods.

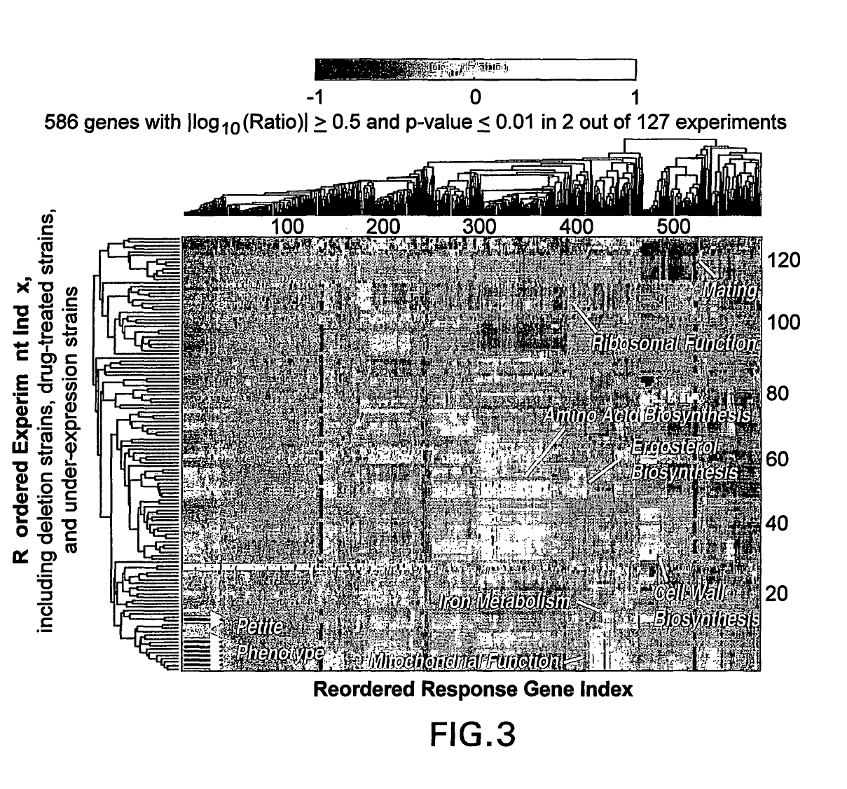

In one embodiment, the intersections of cellular constituent sets are identified by visual inspection of response profile data for a plurality of perturbations. Preferably, such data is re-ordered, according, e.g., to the methods described in Section 5.5.1 and 5.5.3, above, so that co-varying cellular constituents and similar response profiles can be more readily identified. For example, FIG. 3 shows a false color display of a plurality of genetic transcripts (horizontal axis) measured in a plurality of experiments (i.e., response profiles) wherein cells of S. cerevisiae are exposed to a variety of different perturbations as indicated on the vertical axis. Both the cellular constituents and the response profiles have been grouped and re-ordered according to the methods of Sections 5.5.1 and 5.5.3, and those described in U.S. patent application Ser. No. 09/220,142 to Stoughton et al., filed Dec. 23, 1998 (incorporated by reference herein in its entirety), so that the co-varying cellular constituents (i.e., genesets) and similar response profiles can be readily visualized.

In other, more formal quantitative embodiments of the invention, the intersections of cellular constituent sets are preferably identified, e.g., by thresholding the individual response amplitudes of the projected response profiles. In particular, thresholds are set at a detection limit equal to two standard errors of the geneset response, assuming uncorrelated errors in the individual genes, or standard error of ˜0.15 in the log₁₀. With the preferred normalization of the basis vectors (i.e., with |e^{(q)}|=1 for all genesets q), the appropriate threshold for the geneset amplitude is the same as that for individual genes at a particular desired confidence level.

In alternative embodiments, intersections of cellular constituent sets may be identified arithmetically, by replacing significant amplitudes of cellular constituent sets in the projected responses (i.e., those amplitudes which are above the threshold) with values of unity, and replacing amplitudes of cellular constituent sets in the projected responses that are below the threshold with values of zero. The intersection may then be determined by the element-wise product of all project profiles. In particular, in such embodiments the consensus profile consists of those cellular constituent sets whose index is unity after the product operation.

Comparing Response to Consensus Profiles:

Once basis cellular constituent sets have been identified, e.g., according to the methods described in Section 5.5.1 above, projected profiles P may be obtained for any biological response profile p comprising the same cellular constituent as those used to define the basis cellular constituent sets, e.g., according to the methods provided in Section 5.5.4 above. As noted supra, similarities and differences between two or more projected profiles, for example between the projected profile P^{(a)} and P^{(b)}, can be readily evaluated. In preferred embodiments, projected profiles are compared by an objective, quantitative similarity metric S. In one particularly preferred embodiment, the similarity metric S is the generalized cosine angle between the two projected profiles being compared, e.g., between P^{(a)} and P^{(b)}. The generalized cosine angle is a metric well known to those skilled in the art, and is provided, below, in Equation 24:

$${S}_{a , b} = S ( {P}^{( a )} , {P}^{( b )} ) = \frac{{P}^{( a )} \cdot {P}^{( b )}}{\vert {P}^{( a )} ‖ {P}^{( b )} \vert} \qquad ( 24 )$$

In Equation 24, the dot product p^{(a)}·p^{(b)} is defined according to Equation 25:

$${P}^{( a )} \cdot {P}^{( b )} = {\sum }_{q}^{} ( {P}_{q}^{( a )} \times {P}_{q}^{( b )} ) \qquad ( Equation 25 )$$

Likewise, the quantities |P^{(a)}| and |P^{(b)}| are provided according to the equations |P₍ₐ₎|=(P^{(a)}·P₍ₐ₎)^{1/2}, and |P^{(b)}|=(P^{(b)}·P^{(b)})^{1/2}.

In such embodiments, projected profile P^{(a)} is most similar to the projected profile P^{(b)} if S_{a,b} is a maximum. In more detail, S_{a,b} may have a value from −1 to +1. A value of S_{a,b}=+1 indicates that the two profiles are essentially identical; the same cellular constituent effected in P^{(a)} are proportionally effected in P^{(b)}, although the magnitude (i.e., strength) of the two responses may be different. A value of S_{a,b}=−1 indicates that the two profiles are essentially opposites. Thus, although the same cellular constituent sets in P^{(a)} are proportionally effected in P^{(b)}, those sets which increase (e.g., are up-regulated) in P^{(a)} decrease (e.g., are down regulated) in P^{(b)} and vice-versa. Such profiles are said to be “anti-correlated.” Finally, a value of S_{a,b}=0 indicates maximum dissimilarity between the two responses; those cellular constituent sets effected in P^{(a)} are not effected in P^{(b)} and vice-versa.

Projected profiles may also be compared to the consensus profiles P^{(C)} of the present invention. Such comparisons are useful, e.g., to determine whether a particular response profile, e.g., of the biological response to a drug or drug candidate, is consistent with or false short of the consensus profile, e.g., for a class or type of drugs, or for an “ideal” biological response such as one associated with a desired therapeutic effect. Projected profiles may be compared to the consensus profiles of this invention by means of the same methods described supra for comparing projected profiles generally. Thus a give projected profile P^{(a)} may be compared to a consensus profile P^{(C)}, e.g., by evaluating a quantitative similarity metric Sₐ^{(C)}=S(P^{(a)}, P^{(C)}), wherein S(P^{(a)}, P^{(C)}) is defined, e.g. according to Equation 24 above.

The statistical significance of any observed similarity S_{a,b} may be assessed, e.g., using an empirical probability of distribution generated under the null hypothesis of no correlation. Such a distribution may be generated by performing projection and similarity calculations, e.g., according to the above described methods and equations, for many random permutations of the cellular constituent index i in the original unprojected response profile p. Mathematically, such a permutation may be represented by replacing the ordered set {pᵢ} by {p_{II(i)}), where II(i) denotes a permutation of the index i. Preferably, the number of permutations is anywhere from about 100 to about 1000 different random permutations. The probability that the similarity S_{a,b} arises by chance may then be determined from the fraction of the total permutations for which the similarity S_{a,b}^{(permuted)} exceeds the similarity S_{a,b} determined for the original, unpermuted data.

Clustering Projected Profiles:

The present invention also provides methods for clustering and/or sorting projected profiles, e.g., by means of the clustering methods described in Section 5.5.1 and 5.5.3 above, according to their similarity as evaluated, e.g., by a quantitative similarity metric S such as the generalized cosine angle. In a preferred embodiment, the clustering of a projected profile is done using the distance metric given, below, in Equation 26:

I_{a,b}=1−S_{a,b} (26)

In a particularly preferred embodiment of this invention, the projected profiles are clustered or ordered according to their similarity to a consensus profile P_{(C)}, e.g., using the distance metric I=1−S^{(C)}=1−S(P, P^{(C)}), wherein P is the projected response profile to be sorted according to the methods of the present invention.

Such clustering and sorting methods are analogous to the clustering of the original unprojected response profiles described in Section 5.5.3 above. However, the clustering of projected response profiles has the advantages of reduced measurement error effects and enhanced capture of the relevant biology inherent to the projected response profiles.

Removal of Profile Artifacts:

In a preferred embodiment, the projection methods described above can also be used to remove unwanted response components (i.e., “artifacts”) from biological profile (e.g., perturbation response profile) data. Frequently, when such profile data are obtained there are one or more poorly controlled variables which lead to measured patterns of cellular constituents (e.g., measured gene expression patterns) which are, in fact, artifacts of the measurement process and are not part of the actual biological state or response (such as a perturbation response) being measured. Exemplary variables which may produce artifacts in biological profile data include, but are by no means limited to, cell culture density and temperature and hybridization temperature, as well as concentrations of total RNA and/or hybridization reagents.

For example, Di Risi et al. (1997, Science 278:680-686) describe measurements using microarrays of S. cerevisiae cDNA levels during the change from anaerobic to aerobic growth (i.e., the “diauxic shift”). However, if one of two nominally identical cell cultures has unintentionally progressed further into the diauxic shift than the other, their expression ratios will reflect that transcriptional changes associated with this shift. Such artifacts potentially confuse the measurements of the true transcriptional responses being sought. These artifacts may be “projected out” by removing or suppressing their patterns in the data.

In preferred embodiments, the artifact patterns in the data are known. In general, artifact patterns may be determined from any source of knowledge of the genes and relative amplitudes of response associated with such artifacts. For example, the artifact patterns may be derived from experiments with intentional perturbations of the suspected causative variables. In another embodiment, the artifact patterns may be determined from clustering analysis of control experiments where the artifacts arise spontaneously.

In such preferred embodiments, the contribution of known artifacts may be solved for and subtracted from the measured biological profile p={pᵢ}, e.g., by determining the best scaling coefficients αₙ for the contribution of artifact n to the profile. Preferably, the coefficients αₙ are found by determining the values of an which minimize an objective function of the difference between the measured profile and the scaled contribution of the artifacts. For example, the coefficients an may be determined by the least square minimization

$${min}_{{α}_{n}} \{ {\sum }_{i}^{} {( {p}_{i} - {\sum }_{n}^{} {α}_{n} {A}_{n , i} )}^{2} {w}_{i} \} \qquad ( 27 )$$

wherein A_{n,i} is the amplitude of artifact n on the measurement of cellular constituent i. wᵢ is an optional weighting factor selected by a user according to the relative certainty or significance of the measured value of cellular constituent i (i.e., of pᵢ).

The “cleaned” profile p^{(clean)} in which the artifacts are effectively removed, is then given by the equation

$${p}_{i}^{( clean )} = {p}_{i} - {\sum }_{n}^{} {α}_{n} {A}_{n , i} \qquad ( 28 )$$

wherein the coefficients αₙ are determined, e.g., from equation 27 above.

In other embodiments, the profile p may be compared to a library of artifact signatures Aₛ={A_{s,i}} of different severity. In such embodiments, the “cleaned” profile is determined by pattern matching against this library to determine the particular template which has greatest similarity to the profile p. In such embodiments, the cleaned profile is given by pₖ^{(clean)}=pₖ−A_{s,i}, wherein the signature Aₛ is determined, e.g., by solving the equation

$${min}_{s} \{ {\sum }_{i}^{} {( {p}_{i} - {A}_{s , i} )}^{2} {w}_{i} \} \qquad ( 29 )$$

### 5.6 Prediction of Mature Phenotype from Profiles of Immature Phenotypes

One of skill in the art will recognize that the transcript profile of a given genotype changes with developmental stage and tissue type in multi-cellular organisms, as well as with environmental conditions during growth. A compendium is preferably generated under a consistent set of conditions, e.g., corn seedling leaf at 6 days old, grown using a particular nutrient mix and growth temperature. However, some phenotypes of interest, for example the yield of seed products in grains, are manifested by proteins and mechanisms that come into play only at later developmental stages. In one embodiment, profiles are obtained from the appropriate mature tissue and compared to a compendium of landmark profiles from tissue of the same type and level of maturity.

In a preferred embodiment, an immature seedling can be profiled (filled circle in FIG. 1) and the developmental track of its transcriptional response extrapolated, as indicated by the dashed line in FIG. 1. From the measurements performed on other individual phenotypes, it is possible to determine from the profile of the immature seedling what developmental track it will follow. Thus, measurement of the immature profile will predict the mature profile, and the proximity of immature profiles in transcriptional response space indicates eventual proximity of the mature profiles, allowing identification of candidate causative genes based on a compendium of genetic landmark profiles taken from immature phenotypes. Working with profiles of immature phenotypes is advantageous because much less time and cost is required to reach the seedling stage than to grow a plant to maturity and test it for a particular phenotype under particular field conditions. Moreover, seedlings are smaller, require much less space in which to grow, and can more easily be maintained in a controlled environment, such as a greenhouse.

### 5.7 Computer Implementation

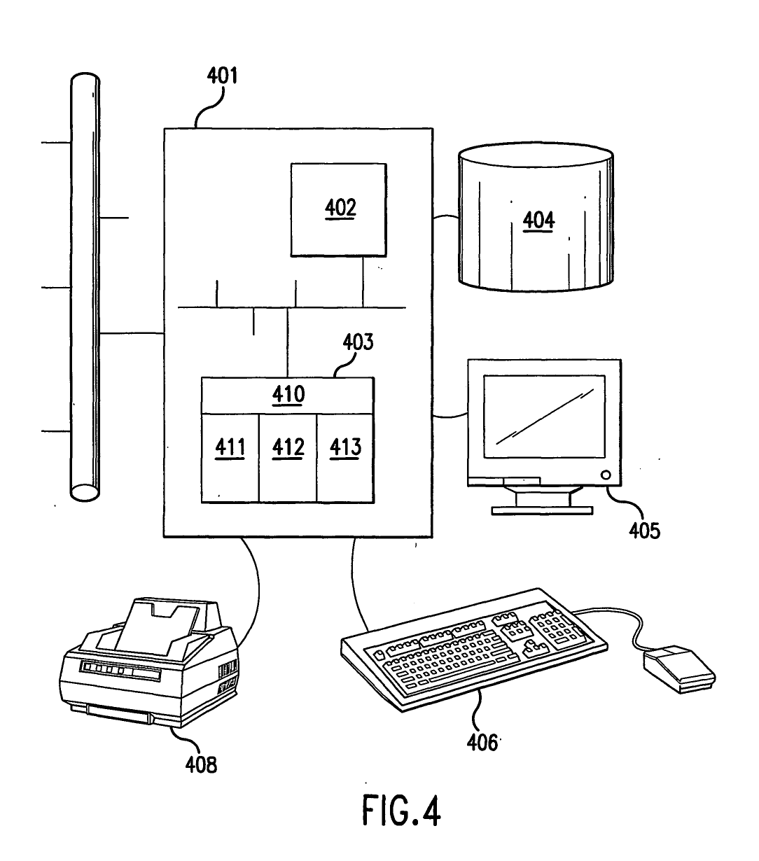

The analytic methods described in the previous subsections can preferably be implemented by use of the following computer systems and according to the following programs and methods. FIG. 4 illustrates an exemplary computer system suitable for implementation of the analytic methods of this invention. Computer system 401 is illustrated as comprising internal components and being linked to external components. The internal components of this computer system include processor element 402 interconnected with main memory 403. For example, computer system 401 can be an Intel Pentium®-based processor of 200 MHz or greater clock rate and with 32 MB or more of main memory.

It is noted that although the present description and figures refer to an exemplary computer system having a memory unit and a processor unit, the computer systems of the present invention are not limited to those consisting of a single memory unit or a single processor unit. Indeed, computer systems comprising a plurality of processor units and/or a plurality of memory units (e.g., having a plurality of SIMMS or DRAMS) are well known in the art. Indeed, such systems are generally recognized in the art as having improved performance capabilities over computer systems that have only a single processor unit or a single memory unit. For example, in one preferred embodiment, computer system 401 is an Alta cluster of nine computers; a head “node” and eight sibling “nodes,” each having an i686 central processing unit (“CPU”). In addition, the Alta cluster comprises 128 Mb of random access memory (“RAM”) on the head node and 256 Mb of RAM on each of the eight sibling nodes. Nevertheless and as the skilled artisan readily appreciates, as such computer systems relate to the present invention, a computer system that has a plurality of memory units and/or a plurality or processor units is, in fact, substantially equivalent to the exemplary computer system depicted in FIG. 4 and having only a single processor and a single memory unit.

The external components include mass storage 404. This mass storage can be one or more hard disks which are typically packaged together with the processor and memory. Such hard disks are typically of 1 Gb or greater storage capacity and more preferably having at least 6 Gb of storage capacity. For example, in the preferred embodiment described above each node of the Alta cluster comprises a hard drive. Specifically, the head node has a hard drive with 6 Gb of storage capacity whereas each sibling node has a hard drive with 9 Gb of storage capacity. Other external components include user interface device 405, which can be a monitor and a keyboard together with a pointing device 406 such as a “mouse” or other graphical input device. Typically, the computer system is also linked to a network link 407, which can be, e.g., part of an Ethernet link to other local computer systems, remote computer systems, or wide area communication networks such as the Internet. For example, each computer system in the preferred Alta cluster of computers described above is connected via an NFS network. This network link allows the computer systems in the cluster to share data and processing tasks with one another.

Loaded into memory during operation of this system are several software components, which are both standard in the art and special to the instant invention. These software components collectively cause the computer system to function according to the methods of the invention. The software components are typically stored on mass storage 404. Software component 410 represents an operating system, which is responsible for managing the computer system and its network interconnections. The operating system can be, for example, of the Microsoft Windows™ family, such as Windows 98, Window 95 or Windows NT. Alternatively, the operating system can be a Macintosh operating system, a UNIX operating system or the LINUX operating system. Software component 411 represents common languages and functions conveniently present in the system to assist programs implementing the methods specific to the present invention. Languages that can be used to program the analytic methods of the invention include, for example, UNIX or LINUX shell command languages such as C, and C++; PERL; FORTRAN; HTML; and JAVA. The methods of the present invention can also be programmed or modeled in mathematical software packages which allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Chapaign, Ill.) or S-Plus from Math Soft (Seattle, Wash.). Accordingly, software component 412 represents analytic methods of the present invention as programmed in a procedural language or symbolic package. In a preferred embodiment, the computer system also contains a database 413 of landmark expression profiles.

In an exemplary implementation, to practice the methods of the present invention, a user first loads expression profile data into the computer system 401. These data can be directly entered by the user from monitor 405 and keyboard 406, or from other computer systems linked by network connection 407, or on removable storage media such as a CD-ROM or floppy disk (not illustrated) or through the network (407). Next the user causes execution of expression profile analysis software 412 which performs the steps of comparing the expression profile to the database 413 of landmark profiles.

In another exemplary implementation, a user first loads expression profile data into the computer system. Geneset profile definitions are loaded into the memory from the storage media (404) or from a remote computer, preferably from a dynamic geneset database system, through the network (407). Next the user causes execution of projection software which performs the steps of converting the expression profile to a projected expression profile. Next, the user causes the execution of comparison software which performs the steps of objectively comparing the projected expression profile to a database of landmark projected expression profiles.

In yet another exemplary implementation, a user first loads a projected profile into the memory. The user then causes the loading of a reference profile from the database of landmark profiles into the memory. Next, the user causes the execution of comparison software which performs the steps of objectively comparing the profiles.

In exemplary implementation, the computer system is capable of determining one or more candidate genes, or their encoded RNAs or proteins, responsible for a phenotype of interest displayed by a cell or organism, and comprises: (a) one more memory units; and (b) one or more processor units interconnected with the one or more memory units, wherein the one or more memory units encodes one or more programs causing the one or more processor units to perform a method comprising comparing a first profile or a predicted profile derived therefrom to a database comprising a plurality of landmark profiles to determine the one or more landmark profiles most similar to said first profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene; and wherein the genes perturbed in the one or more landmark profiles determined to be most similar are those candidate genes responsible for the phenotype of interest.

In another exemplary implementation, the computer system is capable of determining if a desired genotype associated with a phenotype of interest is present in a cell type or organism, and comprises: (a) one or more memory units; and (b) one or more processor units interconnected with the one or more memory units, wherein the one or more memory units encodes one or more programs causing the one or more processor units to perform a method comprising comparing a first profile or a predicted profile derived therefrom to a database comprising a plurality of landmark profiles to determine the one or more landmark profiles among those profiles known to be indicative of the presence or absence of a genotype associated with the phenotype of interest most similar to said first profile most similar to said first profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene; and wherein the genotype indicated in the one or more landmark profiles determined to be most similar is indicative of the phenotype of interest.

In yet another exemplary implementation, the computer system is capable of determining if a genotype associated with an undesirable phenotype is present in a cell type or organism, and comprises: (a) one or more memory units; and (b) one or more processor units interconnected with the one or more memory units, wherein the one or more memory units encodes one or more programs causing the one or more processor units to perform a method comprising comparing a first profile or a predicted profile derived therefrom to a database comprising a plurality of landmark profiles to determine the one or more landmark profiles among those profiles known to be indicative of the presence or absence of a genotype associated with the undesirable phenotype most similar to said first profile most similar to said first profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene; and wherein the genotype indicated in the one or more landmark profiles determined to be most similar is indicative of the undesirable phenotype.

In an exemplary implementation, the computer program product for use in conjunction with a computer having one or more memory units and one or more processor units comprises a computer readable storage medium having a computer program mechanism encoded thereon, wherein said computer program mechanism may be loaded into the one or more memory units of a computer and cause the one or more processor units of the computer to execute the step of comparing a first profile or a predicted profile derived therefrom to a database comprising a plurality of landmark profiles to determine the one or more landmark profiles among those profiles known to be indicative of the presence or absence of a genotype associated with the phenotype of interest most similar to said first profile most similar to said first profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### 5.8 Analytic Kit Implementation

In a preferred embodiment, the methods of this invention can be implemented by use of kits for determining the state of a biological sample. Such kits contain microarrays, such as those described in Subsections below. The microarrays contained in such kits comprise a solid phase, e.g., a surface, to which probes are hybridized or bound at a known location of the solid phase. Preferably, these probes consist of nucleic acids of known, different sequence, with each nucleic acid being capable of hybridizing to an RNA species or to a cDNA species derived therefrom. In particular, the probes contained in the kits of this invention are nucleic acids capable of hybridizing specifically to nucleic acid sequences derived from RNA species that are known to increase or decrease in a phenotype that is determined by the kit. The probes contained in the kits of this invention preferably substantially exclude nucleic acids that hybridize to RNA species that are not increased or decreased in a phenotype that is determined by the kit.

In particular, kits can be used to assay a phenotype, i.e., by determining the expression profile of a cell having a known phenotype and comparing the profile to a compendium of landmark profiles from cells having a known genotype in order to relate the phenotype to genotype. Alternatively, the expression profile of a cell having an unknown phenotype can be determined using the kits of the invention and its phenotype predicted by comparing the profile to a compendium of landmark profiles from cells having a known genotype and a known phenotype.

Alternative kits for implementing the analytic methods of this invention will be apparent to one of skill in the art and are intended to be comprehended within the accompanying claims.

### 5.9 Methods for Determining Biological Response

In general, the profiling methods of the present invention can be performed using any probe or probes that comprise a polynucleotide sequence and which are immobilized to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial sequences of genomic DNA, cDNA, or mRNA sequences extracted from cells. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences that are attached to a nitrocellulose or nylon membrane or filter. Such hybridization probes are well known in the art (see, e.g., Sambrook et al., Eds., 1989, Molecular Cloning: A Laboratory Manual, 2nd ed., Vol. 1-3, Cold Spring Harbor Laboratory, Cold Spring Harbor, N.Y.). Alternatively, the solid support or surface may be a glass or plastic surface.

### 5.9.1 Microarrays Generally

This invention is particularly useful for the analysis of gene expression profiles in order to determine the genotype of a cell. Some embodiments of this invention are based on measuring the transcriptional state of a cell.

The transcriptional state can be measured by techniques of hybridization to microarrays of probes consisting of a solid phase on the surface of which are immobilized a population of polynucleotides, such as a population of DNA or DNA mimics, or, alternatively, a population of RNA or RNA mimics. The solid phase may be a non-porous or, optionally, a porous material such as a gel. In various alternative embodiments, microarrays can be employed for analyzing aspects of the biological state of a cell other than the transcriptional state, such as the translational state, the activity state, or mixed aspects.

In preferred embodiments, a microarray comprises a support or surface with an ordered array of binding (e.g., hybridization) sites or “probes” for products of many of the genes in the genome of a cell or organism, preferably most or almost all of the genes. Preferably the microarrays are addressable arrays, preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics: The arrays are reproducible, allowing multiple copies of a given array to be produced and easily compared with each other. Preferably, microarrays are made from materials that are stable under binding (e.g., nucleic acid hybridization) conditions, and include large nylon arrays, such as those sold by Research Genetics. The microarrays are preferably small, e.g., between 5 cm² and 25 cm², preferably between 12 cm² and 13 cm². However, larger arrays are also contemplated and may be preferable, e.g., for use in screening and/or signature chips comprising a very large number of distinct oligonucleotide probe sequences. Preferably, a given binding site or unique set of binding sites in the microarray will specifically bind (e.g., hybridize) to the product of a single gene in a cell (e.g. to a specific mRNA, or to a specific cDNA derived therefrom). However, in general other, related or similar sequences will cross hybridize to a given binding site. Although there may be more than one physical binding site per specific RNA or DNA, for the sake of clarity the discussion below will assume that there is a single, completely complementary binding site.

The microarrays of the present invention include one or more test probes, each of which has a polynucleotide sequence that is complementary to a subsequence of RNA or DNA to be detected. Each probe preferably has a different nucleic acid sequence, and the position of each probe on the solid surface is preferably known. Indeed, the microarrays are preferably addressable arrays, and more preferably are positionally addressable arrays. Specifically, each probe of the array is preferably located at a known, predetermined position on the solid support such that the identity (i.e., the sequence) of each probe can be determined from its position on the array (i.e., on the support or surface).

Preferably, the density of probes on a microarray is about 100 different (ie., non-identical) probes per 1 cm² or higher. More preferably, a microarray of the invention will have at least 550 different probes per 1 cm², at least 1,000 different probes per 1 cm², at least 1,500 different probes per 1 cm² or at least 2,000 different probes per 1 cm². In a particularly preferred embodiment, the microarray is a high density array, preferably having a density of at least about 2,500 different probes per 1 cm². The microarrays of the invention therefore preferably contain at least 2,500, at least 5,000, at least 10,000, at least 15,000, at least 20,000, at least 25,000, at least 50,000, at least 55,000, at least 100,000 or at least 150,000 different (i.e., non-identical) probes per 1 cm².

In specific embodiments, the density of probes on a microarray is between about 100 and 1,000 different (i.e., non-identical) probes per 1 cm², between 1,000 and 5,000 different probes per 1 cm², between 5,000 and 10,000 different probes per 1 cm², between 10,000 and 15,000 different probes per 1 cm², between 15,000 and 20,000 different probes per 1 cm², between 50,000 and 100,000 different probes per 1 cm², between 100,000 and 500,000 different probes per 1 cm², or more than 500,000 different (i.e., non-identical) probes per 1 cm².

In one embodiment, the microarray is an array (i.e., a matrix) in which each position represents a discrete binding site for a product encoded by a gene (i.e., an mRNA or a cDNA derived therefrom), and in which binding sites are present for products of most or almost all of the genes in the organism's genome. For example, the binding site can be a DNA or DNA analogue to which a particular RNA can specifically hybridize. The DNA or DNA analogue can be, e.g., a synthetic oligomer, a full-length cDNA, a less-than full length cDNA, or a gene fragment. Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often to at about 75%, more often to at least about 85%, even more often to about 90%, and still more often to at least about 99%. Alternatively, however, “picoarrays” may also be used. Such arrays are microarrays which contain binding sites for products of only a limited number of genes in the target organism's genome. Generally, a picoarray contains binding sites corresponding to fewer than about 50% of the genes in the genome of an organism.

Preferably, the microarray has binding sites for genes associated with one or more biological pathways responsible for producing a phenotype of interest. A “gene” is typically identified as the portion of DNA that is transcribed by RNA polymerase. Thus, a gene may include a 5′ untranslated region (“UTR”), introns, exons and a 3′ UTR. Thus, a gene comprises at least 25 to 100,000 nucleotides from which a messenger RNA is transcribed in the organism or in some cell in a multicellular organism. The number of genes in a genome can be estimated from the number of mRNAs expressed by the organism, or by extrapolation from a well characterized portion of the genome. When a genome having few introns of an organism of interest, such as yeast, has been sequenced, the number of open reading frames (“ORFs”) can be determined and mRNA coding regions identified by analysis of the DNA sequence. For example, the genome of Saccharomyces cerevisiae has been completely sequenced, and is reported to have approximately 6275 ORFs longer than 99 amino acids. Analysis of these ORFs indicates that there are 5885 ORFs that are likely to encode protein products (Goffeau et al., 1996, Science 274:546-567). In contrast, the human genome is estimated to contain approximately 10⁵ genes.

### 5.9.2 Preparation of Probes for Microarrays

As noted above, the “probe” to which a particular polynucleotide molecules specifically hybridizes according to the invention is a complementary polynucleotide sequence. In one embodiment, the probes of the microarray comprise nucleotide sequences greater than about 250 bases in length corresponding to one or more genes or gene fragments. For example, the probes may comprise DNA or DNA “mimics” (e.g., derivatives and analogues) corresponding to at least a portion of each gene in an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of gene segments from genomic DNA, cDNA (e.g., by RT-PCR), or cloned sequences. PCR primers are preferably chosen based on known sequence of the genes or cDNA that result in amplification of unique fragments (i.e., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 50,000 bases, and usually between 300 bases and 1000 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14:5399-5407; McBride et at, 1983, Tetrahedron Lett. 24:246-248). Synthetic sequences are typically between about 15 and about 500 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Eghohn et al., 1993, Nature 363:566-568; U.S. Pat. No. 5,539,083).

In alternative embodiments, the hybridization sites (i.e., the probes) are made from plasmid or phage clones of genes, cDNAs (e.g., expressed sequence tags), or inserts therefrom (Nguyen et al., 1995, Genomics 29:207-209).

### 5.9.3 Attaching Probes to the Solid Surface

The probes are attached to a solid support or surface, which may be made, e.g. from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, 1995, Science 270:467-470. This method is especially useful for preparing microarrays of cDNA (See also, DeRisi et al, 1996, Nature Genetics 14:457-460; Shalon et al, 1996, Genome Res. 6:639-645; and Schena et al, 1995, Proc. Natl. Acad. Sci. U.S.A. 93:10539-11286).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al, 1994, Proc. Natl. Acad. Sci. USA. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et at., Biosensors & Bioelectronics 11:687-690). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al, supra) could be used. However, as will be recognized by those skilled in the art, very small arrays will frequently be preferred because hybridization volumes will be smaller.

In a particularly preferred embodiment, microarrays of the invention are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioeletronics 11:687-690; Blanchard, 1998, in Synthetic DNA Arrays in Genetic Engineering, Vol. 20, J. K. Setlow, Ed., Plenum Press, New York at pages 111-123; U.S. Pat. No. 6,028,189 to Blanchard. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes).

### 5.9.4 Target Polynucleotide Molecules

As described, supra, the polynucleotide molecules which may be analyzed by the present invention may be from any source, including naturally occurring nucleic acid molecules, as well as synthetic nucleic acid molecules. In a preferred embodiment, the polynucleotide molecules analyzed by the invention comprise RNA, including, but by no means limited to, total cellular RNA, poly(A)⁺ messenger RNA (mRNA), fraction thereof, or RNA transcribed from cDNA (i.e., cRNA; see, e.g., Linsley & Schelter, U.S. patent application Ser. No. 09/411,074, filed Oct. 4, 1999). Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299). In an alternative embodiment, which is preferred for S. cerevisiae, RNA is extracted from cells using phenol and chloroform, as described in Ausubel et al. (Ausubel et al., eds., 1989, Current Protocols in Molecular Biology, Vol III, Green Publishing Associates, Inc., John Wiley & Sons, Inc., New York, at pp. 13.12.1-13.12.5). Poly(A)⁺ RNA is selected by selection with oligo-dT cellulose. Cells of interest include wild-type cells and mutant cells.

In one embodiment, RNA can be fragmented by methods known in the art, e.g. by incubation with ZnCl₂, to generate fragments of RNA. In one embodiment, isolated mRNA can be converted to antisense RNA synthesized by in vitro transcription of double-stranded cDNA in the presence of labeled dNTPs (Lockhart et al., 1996, Nature Biotechnology 14:1675).

In other embodiments, the polynucleotide molecules to be analyzed may be DNA molecules such as fragmented genomic DNA, first strand cDNA which is reverse transcribed from mRNA, or PCR products of amplified mRNA or cDNA.

Labeled cDNA is prepared from mRNA by oligo dT-primed or random-primed reverse transcription, both of which are well known in the art (see, e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Expression monitoring by hybridization to high-density oligonucleotide arrays, Nature Biotech. 14:1675, which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs or by nonenzymatic conjugation of NHS-ester dyes to aminoallyl-modified nucleotides), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perlin Elmer Cetus), Cy2, Cy3, Cy3.5, Cy5, Cy5.5, Cy7, Fluor X (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., 1995, High density cDNA filter analysis: a novel approach for large-scale, quantitative analysis of gene expression, Gene 156:207; Pietu et al., 1996, Novel gene transcripts preferentially expressed in human muscles revealed by quantitative hybridization of a high density cDNA array, Genome Res. 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™II, LTT Inc.) at 42° C. for 60 min. (Schena et al, 1995, Science 270:467-470).

5.9.5 Hybridization to Microarrays

As described supra, nucleic acid hybridization and wash conditions are chosen so that the polynucleotide molecules to be analyzed by the invention (referred to herein as the “target polynucleotide molecules) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S. 93:10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

### 5.9.6 Signal Detection and Data Analysis

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (ie., capable of specifically binding the product of the gene) that is not transcribed in the cell will have little or no signal (e.g., fluorescent signal), and a gene for which the encoded mRNA is prevalent will have a relatively strong signal.

In preferred embodiments, cDNAs from two different cells are hybridized to the binding sites of the microarray. In the case of the instant invention, one cell is a wild-type cell and another cell of the same type has a mutation in a specific gene. The cDNA derived from each of the two cell types are differently labeled so that they can be distinguished. In one embodiment, for example, cDNA from a cell with a mutation in a specific gene is synthesized using a fluorescein-labeled dNTP, and cDNA from a second, wild-type cell is synthesized using a rhodamine-labeled dNTP. When the two cDNAs are mixed and hybridized to the microarray, the relative intensity of signal from each cDNA set is determined for each site on the array, and any relative difference in abundance of a particular mRNA is thereby detected.

In the example described above, the cDNA from the mutant cell will fluoresce green when the fluorophore is stimulated, and the cDNA from the wild-type cell will fluoresce red. As a result, when the mutation has no effect, either directly or indirectly, on the relative abundance of a particular mRNA in a cell, the mRNA will be equally prevalent in both cells, and, upon reverse transcription, red-labeled and green-labeled cDNA will be equally prevalent. When hybridized to the microarray, the binding site(s) for that species of RNA will emit wavelength characteristic of both fluorophores. In contrast, when the either directly or indirectly increases the prevalence of the mRNA in the cell, the ratio of green to red fluorescence will increase. When the mutation decreases the mRNA prevalence, the ratio will decrease.

The use of a two-color fluorescence labeling and detection scheme to define alterations in gene expression has been described, e.g., in Shena et al., 1995, Science 270:467-470. An advantage of using cDNA labeled with two different fluorophores is that a direct and internally controlled comparison of the mRNA levels corresponding to each arrayed gene in two cell genotypes can be made, and variations due to minor differences in experimental conditions (e.g., hybridization conditions) will not affect subsequent analyses.

In a preferred embodiment, the fluorescent labels in two-color differential hybridization experiments are reversed to reduce biases peculiar to individual genes or array spot locations, and consequently, to reduce experimental error. In other words, it is preferable to first measure gene expression with one labeling (e.g., labeling wild-type cells with a first fluorophore and mutant cells with a second fluorophore) of the mRNA from the two cells being measured, and then to measure gene expression from the two cells with reversed labeling (e.g., labeling wild-type cells with the second fluorophore and mutant cells with the first fluorophore).

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by alterations in the genotype of a cell.

According to the method of the invention, the relative abundance of an mRNA in two cells or cell lines is scored as a perturbation and its magnitude determined (i.e., the abundance is different in the two sources of mRNA tested) or as not perturbed (i.e., the relative abundance is the same). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as a perturbation. Present detection methods allow reliable detection of difference of an order of about 3-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

### 6. EXAMPLES

The following example is presented by way of illustration of the previously described invention and is not limiting of that description.

### 6.1 Example 1

### Generation of Transcript Profiles for A Set of 186 Deletion Strains In Yeast

### 6.1.1 Materials and Methods

### Construction and Growth of Yeast Strains

Deletion strains were constructed as described in Winzler et al. (1999) Science 285:901-06 using a polymerase chain reaction (PCR)-mediated gene disruption strategy that exploits the high rate of homologous recombination in yeast. Groups of 20 mutant strains were streaked to single colonies from glycerol stocks frozen at −80° C. onto fresh plates containing yeast extract, peptone and dextrose (“YPD plates”). For each group of 20 mutants, a new plate of the wild type strain was also streaked. Plates were incubated in a 30° C. incubator for 40-60 hrs, until well-isolated colonies reached a size of approximately 1-2 mm. Plates were stored at 4° C. wrapped in Parafilm®.

The mutants and wild-type control were grown from colonies inoculated into sterile liquid synthetic complete (“SC”) media from the same preparation batch. For each set of 20 mutants, four independent wild-type colonies (A, wild-type and B-D, wild-type controls) were picked. One ml of SC medium was aliquoted into 1.5 ml tubes and primary starting cultures for each mutant and for wild-type colonies A-D were begun by picking a well-isolated 1-2 mm colony, inoculating 1 ml of SC media and vortexing each tube. For overnight cultures, two dilutions of each mutant and of wild-type colonies B-D were made by inoculating 50 μor 150 μl of the primary starting cultures into 5 ml SC medium. For colony A, dilutions of the primary starting culture were made by inoculating either 250 μl or 750 μl into 100 ml of SC media. Cultures were placed in a 30° C. incubator overnight (14-16 hrs) with mixing. Cell densities were assayed by taking duplicate absorbance readings for 200 μl of culture at 630 nm (A₆₃₀) in an ELₓ800 96-well plate spectrophotometer. Cultures having a density of between 0.2 and 0.5 were used.

100 ml of SC media was measured into sterile 250 ml flasks (one for each mutant and for each wild-type control colony B-D, and an equal number for a corresponding wild-type colony A culture for each of these) with foil caps, and the flasks were placed in an incubator/shaker to pre-warm at 30° C. The amount of overnight culture necessary to innoculate a 100 ml culture to a target reading of 0.01 A₆₃₀ units was calculated and was added to each flask. For wild-type, this dilution reached a target cell density of 0.16 A₆₃₀ units at harvest when grown shaking (300 rpm) at 30° C. for approximately 6 hrs. For slower growing mutants, wild-type and mutant were harvested at similar cell densities by starting the wild-type at a lower density. All starting and harvest times and densities were recorded to determine the growth rate for each mutant.

Wild-type and mutant cultures were harvested in log phase growth as determined by an N30 reading of 0.15 to 0.20 (6-8×10⁶ cells/ml) taken as described above. Prior to harvest, each mutant and colony B-D culture was assigned to a wild-type colony A counterpart to be carried through all the way to hybridization. Processing of each mutant and each of colonies B-D in the subsequent steps was done in parallel with processing of its wild-type counterpart. All harvesting steps were done as quickly as possible to minimize the time in which changes in gene expression could occur. Flasks were removed from the shaker in mutant/wild-type pairs (and wild-type/colony B-D wild-type controls), no more than 6 cultures at a time. Cultures were quickly transferred to 50 ml conical tubes (2 tubes per 100 ml culture) and immediately spun at 3000 rpm in a room temperature tabletop swinging bucket centrifuge for 2 minutes. Supernatant was poured off and tubes were inverted on Parafilm® or paper towels. After the tubes were drained, excess liquid was removed by flicking each tube twice, and tubes were re-capped. Four tubes were simultaneously frozen by immersing bottoms in liquid nitrogen (approximately 5 secs) and were then transferred to −80° C. The procedure was repeated until all cultures were harvested.

Preparation of Total RNA

Cells were lysed as follows. Frozen cell pellets (in 50 ml tubes, two tubes per mutant or wild-type cell pellet) were removed from the −80° C. freezer and tubes were uncapped. To each tube was added an RNase-free solution of 350 μl of REB/SDS buffer (0.2 M Tris pH 7.6, 0.5 M NaCl, 10 mM EDTA, 1% SDS) and 350 μl of 1:1 phenol:chloroform, tubes were re-capped, vortexed for 5 seconds, and transferred to a wire rack in a 65° C. water bath for 1 minute. Tubes were then vortexed for 5 seconds, incubated at 65° C. for 5 minutes, vortexed again for 5 seconds, removed from the water bath, and vortexed again for 5 seconds. Duplicate samples were combined into labeled microcentrifuge tubes. These were mixed by inversion several times, and then spun for 10 minutes in a microcentrifuge at 14,000 rpm. 600 μl of supernatant from each tube was combined with 600 μl of REB/SDS/phenol-chloroform in a microcentrifuge tube. Tubes were mixed by inversion several times, vortexed for 1 minute, and spun for 10 minutes in a microcentrifuge at 14,000 rpm. 500 μl of supernatant from each tube was aliquoted into a new microcentrifuge tube and 500 μl isopropanol were added. Tubes were mixed by inverting several times until Schlering stops. A precipitate formed almost immediately. Tubes were incubated for at least 10 min. at −80° C. and were spun for more than 30 min. at 16,000 rpm in an SS-20 rotor at 4° C. Supernatant was removed, and pellets were resuspended in 960 μl TE (10 mM Tris pH 7.6, 0.1 mM EDTA) and then incubated at 65° C. for 10 min. 211 of each sample were diluted into 800 μl of TE and the absorbance ratio A₂₆₀/A₂₈₀ was read to determine total RNA yield.

Purification of poly-A RNA

In order to prepare total RNA for purification on an oligo-dT column, 600 μl of total RNA from each sample was placed in a microcentrifuge tube, heated to 70° C. for 10 min., snap-cooled by placing on ice for at least 5 min., and then diluted with 600 μl Rnase-free 2× loading buffer (40 mM Tris pH 7.6, 1 M NaCl, 2 mM EDTA, 0.2% N-lauryl-sarcosine, sodium salt, “SLS”) just prior to column loading. Each 1200 μl sample was loaded onto a 0.6 g oligo-dT cellulose column and was allowed to run through the column until dripping stopped (5 min.). The flow-through was then reloaded twice more onto the column without additional heating. Columns were washed two times with 1.5 ml 1×loading buffer (20 mM Tris pH 7.6, 0.5 M NaCl, 1 mM EDTA, 0.1% SLS), then with 0.4 ml of middle wash buffer (20 mM Tris pH 7.6, 150 mM NaCl, 1 mM EDTA, 0.1% SLS), and eluted three times with 320 μl of elution buffer (10 mM Tris pH 7.6, 0.1 mM EDTA, 0.1% SLS) heated to 70° C. Columns were then regenerated with washes of 1.5 ml 1 M NaOH, 1.5 ml DEPC H₂O, and 1.5 ml 1×loading buffer.

Eluates were heated to 70° C. and snap-cooled, and 240 μl 5× loading buffer (100 mM Tris, pH 7.6, 2.5 M NaCl, 5 mM EDTA, 0.5% SLS) were added to the 1.2 ml eluates. Eluates were bound to the columns and washed with loading buffer and middle wash, as above. Columns were eluted with 250 μl of elution buffer heated to 70° C.

Eluates were transferred into microcentrifuge tubes and ethanol precipitated by adding 50 μl 3 M NaOAc, pH 5.2, 4 μl linear acrylamide (5 μg/μl), and 1.1 ml ethanol. Tubes were incubated overnight at −80° C., and then spun for more than 30 min. at 16,000 rpm in an F20/micro rotor at 4° C. Supernatant was removed from the tubes, pellets were air-dried for 15 min., and 20 μl TE/0.1% SLS was added to each pellet. 2 μl of each sample was diluted in 100 μl TE, and the A₂₆₀/A₂₈₀ was read.

Reverse Transcription

Reverse transcription was performed in a 96-well plate. Each RNA sample was reverse transcribed in two separate reactions (one for each of two fluorophore labels in order to perform a reverse labeling experiment, see above). For each reaction, 2 μl of oligo-dT primer (2.5 μg/μl) and 2 μg of poly-A mRNA in 15.8 μl of DEPC-H₂O were added to each well and mixed. The PCR machine was programmed to run the following program: 70° C. for 10 min.; 4° C. for 10 min.; 42° C. for 2 hrs.; 65° C. for 30 min.; 4° C., hold. After all samples were pipetted into the plate, it was transferred into the PCR machine and the program was started in order to denature the samples. Pre-mix for reverse transcription was made by mixing 720 μl 5× reaction buffer, 144 μl 25×aa-dUTP/dNTP labeling mix, 360 μl DTT, 200 μl Superscript II reverse transcriptase and 40 μl H₂O. After the PCR machine reached 42° C., 12.2 μl of pre-mix were added to each sample and the samples were left to incubate at 42° C. for 115 min., at which point 15 μl of 0.5 M NaOH/0.25 M EDTA were added to each sample in order to hydrolyze the RNA. The samples were then allowed to incubate for 20 min. at 65° C. The plate was removed from the PCR machine, and 20 μL1 of 1 M Tris pH 7.5 was added to each well in order to neutralize the reaction.

cDNA was purified using Microcon-30 microconcentrators and washing each sample with 1,600 μl H₂O in a total of three spins in a microcentrifuge at 12,000 rpm. When the final volume reached 5-10 μl, samples were recovered and dried under vacuum (Speed vac for 45 min.-1 hr. under medium heat). 4.5 μl H₂O was added to each sample in preparation for labeling.

Post-Synthetic Labeling of Amino-Allyl cDNA

One of the two identical cDNA samples (see above) was labeled with the Cy3 dye, while the other was labeled with the Cy5 dye. Monofunctional N-hydroxysuccinimide ester derivatives of Cy3 and Cy5 dyes were prepared by dissolving one Cy3 or Cy5 monoreactive dye pack (Amersham) in 13 μl DMSO, adding 27 μl 2×bicarbonate buffer (1 pellet to 25 ml H₂O and 125 μl 37% HCl), mixing quickly and pipetting 4.5 μl into each of 8 purified cDNA samples. The coupling reaction was allowed to proceed for 1 hour in the dark at room temperature. The reaction was then stopped by adding 4.5 μl 4 M hydroxylamine for 10 min. Appropriate wild-type and mutant cDNA samples (differently labeled) were combined and purified using a Qiaquick PCR purification kit. Samples were washed with 200 μl H₂O and concentrated using a microcon-30 microconcentrator (10 min. spin at 12,000 rpm) in preparation for hybridization, and the final volume of each sample was adjusted to 20 μl with H₂O.

Array Hybridization

To 14.4 μl of each sample of combined Cy3/Cy5 labeled cDNA was added 2.8 μl 20×SSC (3 M NaCl, 0.3 M sodium citrate) and 1.4 μl of 10 mg/ml poly-A DNA as a nonspecific blocker. Labeled sample was filtered through a Millipore 0.45 μm membrane by spinning the sample at 10,000 rpm for 1 minute (20 μl yield). 0.4 μl of 10% SDS was added to PCR tubes, and filtered samples were transferred to the tubes. The final hybridization solution contained 3×SSC (0.45 M NaCl, 45 mM sodium citrate), 0.75 μg/μl poly-A DNA and 0.2% SDS. Samples were then denatured by heating to 100° C. in a PCR machine with a heated lid for 2 min. Probes were cooled with the heated lid in place for 34 minutes to minimize evaporation.

Array slides were placed in hybridization chambers, and a 10 μl drop of 3×SSC was added to the bottom of each slide. 20 μl of labeled cDNA and 0.4% SDS was added to the top of the array, and a custom glass coverslip was placed on top of the array without air being trapped between it and the array slide. The hybridization chambers were closed and were placed in a 63° C. water bath for a minimum of 6 hrs. After incubation, hybridization chambers were removed from the water bath and coverslips were removed from the array slides by placing each slide in a dish containing primary wash solution (20 ml 20×SSC, 1 ml 10% SDS, 330 ml H₂O). Slides were then placed in a second dish containing primary wash solution. Slides were subsequently washed in secondary wash solution (1 ml 20×SSC, 350 ml H₂O) and dried by spinning at 600 rpm at room temperature for 4 min.

Fabrication and Scanning of Microarrays.

PCR products containing 5′ and 3′ sequences (Research Genetics, Huntsville, Ala.) were used as templates with amino modified forward primers and unmodified reverse primers to amplify 6,056 ORFs from the S. cerevisiae genome. The first pass success rate was 94%. Amplification reactions that gave products of unexpected sizes were excluded from subsequent analysis. ORFs that could not be amplified from purchased templates were amplified from genomic DNA DNA samples from 100 μl reactions were precipitated with isopropanol, resuspended in water, brought to a final concentration of 3×SSC in a total volume of 15 μl, and transferred to 384-well microtiter plates (Genetix Limited, Christchurch, Dorset, England). PCR products were spotted onto 1×3-inch polylysine-treated glass slides by a robot built essentially according to defined specifications (http://cmgm.stanford.edu/pbrown/MGuide). After being printed, slides were processed according to published protocols.

Scanning of Microarrays.

Microarrays were imaged on a prototype multiframe CCD camera in development at Applied Precision (Issaquah, Wash.). Each CCD image frame was approximately 2 mm square. Exposure times of 2 sec in the Cy5 channel (white light through Chroma 618-648 nm excitation filter, Chroma 657-727 nm emission filter) and 1 sec in the Cy3 channel (Chroma 535-560 nm excitation filter, Chroma 570-620 nm emission filter) were done consecutively in each frame before moving to the next spatially contiguous frame. Color isolation between the Cy3 and Cy5 channels was about 100:1 or better. Frames were “knitted” together with software to make the complete images. The intensity of spots (about 100 μm) were quantified from the 10 μm pixels by frame-by-frame background subtraction and intensity averaging in each channel. Dynamic range of the resulting spot intensities was typically a ratio of 1,000 between the brightest spots and the background-subtracted additive error level. Normalization between the channels was accomplished by normalizing each channel to the mean intensities of all genes. This procedure is nearly equivalent to normalization between channels using the intensity ratio of genomic DNA spots, but is possibly more robust, as it is based on the intensities of several thousand spots distributed over the array.

### 6.1.2 Results and Discussion

FIG. 3 shows a subset of transcript profiles for a set of 186 genetic deletion strains in yeast. The horizontal coordinate is the index of the responding gene, and the vertical coordinate is the index specifying which gene was deleted in the profiled strain. All expression levels are referenced to wild-type expression levels by use of the two-color procedure outlined in detail, above. In order to group co-regulated genes and similar profiles, columns and rows were rearranged using the cluster analysis methods described in Section 5.5 and its subsections, supra, and in U.S. patent application Ser. No. 09/179,569 (filed Oct. 10, 1998), U.S. patent application Ser. No. 09/220,275 (filed Dec. 23, 1998) and PCT International Publication WO 00/24936 published May 4, 2000, which are incorporated herein by reference in their entirety. Experiments having similar gene responses are clustered on the horizontal axis, and experiments having similar profiles for all genes are clustered on the vertical axis using agglomerative hierarchical clustering. Features in the rearranged data, some of which are labeled in FIG. 3, correspond to genesets that are co-regulated and the gene perturbations responsible for the common transcriptional response. For example, the block labeled “mitochondrial function” contains rows corresponding to deletions of genes necessary for the function of mitochondria as well as genes up-regulated (bright areas) as compensatory responses to loss of function.

Along the left edge of FIG. 3 are arrows indicating “petite” phenotypes. These strains exhibited growth colonies that were visibly smaller than average. These phenotypes cluster around the feature associated with mitochondrial loss of function, but also occur elsewhere (around Recorded Deletion Indices 60 and 120). This is an example of a phenotype appearing in multiple, widely-separate locations in transcriptional response space, such as the frost resistant phenotype in FIG. 1. The individual strains whose profiles cluster in the upper group are near each other in transcriptional response space. Considered as unknowns, these phenotypes would have been related to loss of mitochondrial function by the methods of the present invention, and the candidate causative genes for the phenotype would have been those whose deletion profiles clustered closest to the unknown phenotype. Thus, the clustering methods described in U.S. patent application Ser. No. 09/179,569 (filed Oct. 10, 1998), U.S. patent application Ser. No. 09/220,275 (filed Dec. 23, 1998) and PCT International Publication WO 00/24936, published May 4, 2000 are useful for recognizing transcriptional response motifs, such as the “mitochondrial function” block in FIG. 3, which in turn will aid in interpretation of the candidate genes.

In another embodiment, similarity measures such as those shown in Equations 3 and 4 with a threshold minimum similarity score are sufficient to declare the candidate causative genes.

The clustering of profiles on the vertical axis shows that there is some redundancy in the compendium, even for this fairly small fraction of the total genome (186 genes out of approximately 6,000). In this case, the “mitochondrial function” cluster could be represented by any one of the several profiles in the cluster denoted by arrows. For example, it was noted that expressionprofiles generated from strains harboring deletions in the YHR011w, YER050c and YMR293c genes are interleaved in the profile cluster tree with profiles of strains with deletions of known mitochondrial components (such as aep2, msu1, afg3, cem1, imp2 and kim4). Thus, the “mitochondrial function” cluster could be represented by any one of these profiles for some purposes.

### 7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method for determining one or more candidate genes, or their encoded RNAs or proteins, responsible for a phenotype of interest displayed by a cell type or organism, comprising:
(a) determining measured amounts of a plurality of cellular constituents in a first cell of said cell type or of said organism to create a first profile;
(b) comparing said first profile, or a predicted profile derived therefrom, to a database comprising a plurality of landmark profiles to determine the one or more landmark profiles most similar to said first or predicted profile, each landmark profile comprising measured amounts of a plurality of cellular constituents in a second cell of said cell type or type of organism having a perturbation to a known gene or its encoded RNA or protein,
wherein the genes, or their encoded RNAs or proteins, perturbed in the one or more landmark profiles determined in step (b) are those candidate genes responsible for the phenotype of interest.

2. A method for determining one or more candidate genes, or their encoded RNAs or proteins, responsible for a phenotype of interest displayed by a cell type or organism, comprising:
comparing a first profile or a predicted profile derived therefrom to a database comprising a plurality of landmark profiles to determine the one or more landmark profiles most similar to said first or predicted profile;
wherein said first profile comprises measured amounts of a plurality of cellular constituents in a first cell of said cell type or of said organism;
wherein each landmark profile comprises measured amounts of a plurality of cellular constituents in a second cell of said cell type or type of organism having a perturbation to a known gene or its encoded RNA or protein; and
wherein the genes, or their encoded RNAs or proteins, perturbed in the one or more landmark profiles determined to be most similar are those candidate genes responsible for the phenotype of interest.

3. A method for relating the phenotype of a cell type or organism to a genotype, said method comprising:
(a) determining the measured amounts of a plurality of cellular constituents in a first cell of said cell type or of said organism exhibiting a phenotype to create a first profile;
(b) determining the measured amounts of a plurality of cellular constituents in a second cell of said cell type or of said organism having a genetic perturbation to a known gene to create a landmark profile; and
(c) determining the degree of similarity between said first profile and said landmark profile by comparing said degree of similarity between the measured amounts of said pluralities of cellular constituents,
wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

4. A method for relating the phenotype of a cell type or organism to a genotype, said method comprising:
determining the degree of similarity between a first profile and a landmark profile by comparing the degree of similarity between measured amounts of pluralities of cellular constituents, wherein said first profile comprises measured amounts of a plurality of cellular constituents in a first cell of said cell type or of said organism exhibiting a phenotype, and wherein said landmark profile comprises measured amounts of a plurality of cellular constituents in a second cell of said cell type or of said organism having a genetic perturbation to a known gene,
wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

5. The method of claim 1 or 2, wherein the database comprises landmark profiles for perturbations to at least 100 genes, or their encoded RNAs or proteins, in the genome of said cell type or organism.

6. The method of claim 5, wherein the database comprises landmark profiles for perturbations to at least 250 genes, or their encoded RNAs or proteins, in the genome of said cell type or organism.

7. The method of claim 6, wherein the database comprises landmark profiles for perturbations to at least 500 genes, or their encoded RNAs or proteins, in the genome of said cell type or organism.

8. The method of claim 7, wherein the database comprises landmark profiles for perturbations to at least 5,000 genes, or their encoded RNAs or proteins, in the genome of said cell type or organism.

9. The method of claim 8, wherein the database comprises landmark profiles for perturbations to at least 50,000 genes, or their encoded RNAs or proteins, in the genome of said cell type or organism.

10. The method of claim 9, wherein the database comprises landmark profiles for perturbations to at least 100,000 genes, or their encoded RNAs or proteins, in the genome of said cell type or organism.

11. The method of claim 1 or 2, wherein the database comprises landmark profiles for perturbations to at least ¼ of the genes, or their encoded RNAs or proteins, in the genome of a human, a livestock animal or a plant.

12. The method of claim 11, wherein the database comprises landmark profiles for perturbations to at least ½ of the genes, or their encoded RNAs or proteins, in the genome of a human, a livestock animal or a plant.

13. The method of claim 12, wherein the database comprises landmark profiles for perturbations to at least ¾ of the genes, or their encoded RNAs or proteins, in the genome of a human, a livestock animal or a plant.

14. The method of claim 1 or 2, wherein the database comprises landmark profiles for perturbations to at least 2% of the genes, or their encoded RNAs or proteins, in a genome of said cell type or organism.

15. The method of claim 14, wherein the database comprises landmark profiles for perturbations to at least 5% of the genes, or their encoded RNAs or proteins, in a genome of said cell type or organism.

16. The method of claim 15, wherein the database comprises landmark profiles for perturbations to at least 15% of the genes, or their encoded RNAs or proteins, in a genome of said cell type or organism.

17. The method of claim 16, wherein the database comprises landmark profiles for perturbations to at least 40% of the genes, or their encoded RNAs or proteins, in a genome of said cell type or organism.

18. The method of claim 17, wherein the database comprises landmark profiles for perturbations to at least 75% of the genes, or their encoded RNAs or proteins, in a genome of said cell type or organism.

19. The method of claim 1 or 2, wherein said predicted profile is compared to said database, and said first profile is at a first developmental stage or first condition and said predicted profile is at a second, different developmental stage or condition more similar to the developmental stage or condition of said second cell than said first cell.

20. The method of claim 1 or 2 wherein said first profile comprises measured amounts of at least 1,000 cellular constituents, and said landmark profiles each comprise measured amounts of at least 1,000 cellular constituents.

21. The method of claim 20, wherein said first profile comprises measured amounts of at least 10,000 cellular constituents, and said landmark profiles each comprise measured amounts of at least 10,000 cellular constituents.

22. The method of claim 21, wherein said first profile comprises measured amounts of at least 100,000 cellular constituents, and said landmark profiles each comprise measured amounts of at least 100,000 cellular constituents.

23. The method of claim 22, wherein said first profile comprises measured amounts of at least 500,000 cellular constituents, and said landmark profiles each comprise measured amounts of at least 500,000 cellular constituents.

24. The method of claim 1, 2, 3 or 4 wherein the measured amounts of the pluralities of cellular constituents are determined by a method comprising converting expression data into expression values of a plurality of sets of co-varying genes.

25. The method of claim 1, 2, 3 or 4, wherein said measured amounts of the plurality of cellular constituents comprise abundances of a plurality of RNA species present in said first cell type or organism.

26. The method of claim 25, wherein the abundances of said plurality of RNA species are measured by a method comprising contacting a gene transcript array with RNA from said first cell of said cell type or organism, or with cDNA derived therefrom, wherein a gene transcript array comprises a surface with attached nucleic acids or nucleic acid mimics, said nucleic acids or nucleic acid mimics capable of hybridizing with said plurality of RNA species, or with cDNA derived therefrom.

27. The method of claim 1, 2, 3 or 4, wherein the measured amounts of the plurality of cellular constituents comprise abundances of a plurality of protein species present in said first cell type or organism.

28. The method of claim 27, wherein the abundances of said plurality of protein species are measured by a method comprising contacting an antibody array with proteins from said first cell of said cell-type or organism, wherein said antibody array comprises a surface with attached antibodies, said antibodies capable of binding with said plurality of protein species.

29. The method of claim 1, 2, 3 or 4, wherein the measured amounts of the plurality of cellular constituents comprise activities of a plurality of protein species present in said cell-type.

30. The method of claim 1, 2, 3 or 4, wherein the measured amounts of the plurality of cellular constituents in said first cell of said cell type or of said organism are determined in comparison to a wild-type cell of said cell type or of said organism, and wherein the measured amounts of the plurality of cellular constituents in said second cell of said cell type or of said organism are determined in comparison to a wild-type cell of said cell type or of said organism.

31. The method of claim 1, 2, 3 or 4, wherein the measured amounts of the plurality of cellular constituents in said first cell of said cell type or of said organism and measured amounts of the plurality of cellular constituents in said second cell of said cell type or of said organism are absolute amounts of the pluralities of cellular constituents.

32. A method of determining if a genotype associated with a phenotype of interest is present in a cell type or organism, comprising:
(a) determining measured amounts of a plurality of cellular constituents in a first cell of said cell type or organism to create a first profile; and
(b) comparing said first profile to a database comprising a plurality of landmark profiles to determine whether one or more landmark profiles known to be indicative of the presence or absence of a genotype associated with the phenotype of interest is similar to said first profile, each landmark profile comprising measured amounts of a plurality of cellular constituents in a second cell of said cell type or type of organism having a perturbation to a known gene or its encoded RNA or protein,
wherein determining that the landmark profiles known to be indicative of the presence of said genotype are similar to said first profile, is indicative of the presence of said genotype associated with the phenotype of interest in the cell type or organism; and
wherein determining that the landmark profiles known to be indicative of the absence of said genotype are similar to said first profile, is indicative of the absence of said genotype associated with the phenotype of interest in the cell type or organism.

33. A method of determining if a genotype associated with a phenotype of interest is present in a cell type or organism, comprising:
comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and
wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

34. The method of claim 32 or 33 wherein the phenotype is desirable.

35. The method of claim 32 or 33, wherein the phenotype is undesirable.

36. The method of claim 32 or 33, wherein the database comprises landmark profiles for perturbations to at least 100 genes, or their encoded RNAs or proteins, in the genome of said cell type or organism.

37. The method of claim 36, wherein the database comprises landmark profiles for perturbations to at least 250 genes, or their encoded RNAs or proteins, in the genome of said cell type or organism.

38. The method of claim 37, wherein the database comprises landmark profiles for perturbations to at least 5,000 genes, or their encoded RNAs or proteins, in the genome of said cell type or organism.

39. The method of claim 38, wherein the database comprises landmark profiles for perturbations to at least 100,000 genes, or their encoded RNAs or proteins, in the genome of said cell type or organism.

40. The method of claim 32 or 33, wherein the database comprises landmark profiles for perturbations to at least ½ of the genes, or their encoded RNAs or proteins, in the genome of a human, a livestock animal or a plant.

41. The method of claim 40, wherein the database comprises landmark profiles for perturbations to at least ¾ of the genes, or their encoded RNAs or proteins, in the genome of a human, a livestock animal or a plant.

42. The method of claim 32 or 33, wherein the database comprises landmark profiles for perturbations to at least 2% of the genes, or their encoded RNAs or proteins, in a genome of said cell type or organism.

43. The method of claim 42, wherein the database comprises landmark profiles for perturbations to at least 5% of the genes, or their encoded RNAs or proteins, in a genome of said cell type or organism.

44. The method of claim 43, wherein the database comprises landmark profiles for perturbations to at least 15% of the genes, or their encoded RNAs or proteins, in a genome of said cell type or organism.

45. The method of claim 44, wherein the database comprises landmark profiles for perturbations to at least 75% of the genes, or their encoded RNAs or proteins, in a genome of said cell type or organism.

46. The method of claim 33, wherein said predicted profile is compared to said database, and said first profile is at a first developmental stage or first condition and said predicted profile is at a second, different developmental stage or condition more similar to the developmental stage or condition of said second cell than said first cell.

47. The method of claim 32 or 33 wherein said first profile comprises measured amounts of at least 1,000 cellular constituents, and said landmark profiles each comprise measured amounts of at least 1,000 cellular constituents.

48. The method of claim 47, wherein said first profile comprises measured amounts of at least 100,000 cellular constituents, and said landmark profiles each comprise measured amounts of at least 100,000 cellular constituents.

49. The method of claim 32 or 33, wherein the measured amounts of the pluralities of cellular constituents are determined by a method comprising converting expression data into expression values of a plurality of sets of co-varying genes.

50. The method of claim 32 or 33, wherein the measured amounts of the plurality of cellular constituents comprise abundances of a plurality of RNA species present in said first cell type or organism.

51. The method of claim 50, wherein the abundances of said plurality of RNA species are measured by a method comprising contacting a gene transcript array with RNA from said first cell of said cell type or organism, or with cDNA derived therefrom, wherein a gene transcript array comprises a surface with attached nucleic acids or nucleic acid mimics, said nucleic acids or nucleic acid mimics capable of hybridizing with said plurality of RNA species, or with cDNA derived therefrom.

52. The method of claim 32 or 33, wherein the measured amounts of the plurality of cellular constituents in said first cell of said cell type or of said organism are determined in comparison to a wild-type cell of said cell type or of said organism, and wherein the measured amounts of the plurality of cellular constituents in said second cell of said cell type or of said organism are determined in comparison to a wild-type cell of said cell type or of said organism.

53. The method of claim 32 or 33, wherein measured amounts of the plurality of cellular constituents in said first cell of said cell type or of said organism and the measured amounts of the plurality of cellular constituents in said second cell of said cell type or of said organism are absolute amounts of the pluralities of cellular constituents.

54. A system for determining one or more candidate genes, or their encoded RNAs or proteins, responsible for a phenotype of interest displayed by a cell or organism, said system comprising:
(a) one or more memory units; and
(b) one or more processor units interconnected with the one or more memory units,
wherein the one or more memory units encodes one or more programs causing the one or more processor units to perform a method comprising
comparing a first profile or a predicted profile derived therefrom to a database comprising a plurality of landmark profiles to determine the one or more landmark profiles most similar to said first or predicted profile;
wherein said first profile comprises measured amounts of a plurality of cellular constituents in a first cell of said cell type or of said organism;
wherein each landmark profile comprises measured amounts of a plurality of cellular constituents in a second cell of said cell type or type of organism having a perturbation to a known gene or its encoded RNA or protein;
and wherein the genes perturbed in the one or more landmark profiles determined to be most similar are those candidate genes responsible for the phenotype of interest.

55. The system of claim 54, wherein the measured amounts of the plurality of cellular constituents in said first cell of said cell type or of said organism are determined in comparison to a wild-type cell of said cell type or of said organism, and wherein the measured amounts of the plurality of cellular constituents in said second cell of said cell type or of said organism are determined in comparison to a wild-type cell of said cell type or of said organism.

56. The system of claim 54, wherein the measured amounts of the plurality of cellular constituents in said first cell of said cell type or of said organism and the measured amounts of the plurality of cellular constituents in said second cell of said cell type or of said organism are absolute amounts of the pluralities of cellular constituents.

57. A system for relating the phenotype of a cell type or organism to a genotype, said system comprising:
(a) one or more memory units; and
(b) one or more processor units interconnected with the one or more memory units,
wherein the one or more memory units encodes one or more programs causing the one or more processor units to perform a method comprising
determining the degree of similarity between a first profile of measured amounts of a plurality of cellular constituents in a first cell of said cell type or of said organism exhibiting a phenotype and a landmark profile of measured amounts of a plurality of cellular constituents in a second cell of said cell type or of said organism having a genetic perturbation to a known gene by comparing said degree of similarity between the measured amounts of said pluralities of cellular constituents,
wherein said degree of similarity between said first profile and said landmark profile indicates the degree of similarity between the genotype resulting in the phenotype of said first cell or organism and the known mutant genotype of said second cell or organism, thereby relating the phenotype of said first cell or organism to the genotype of said second cell or organism.

58. The system of claim 57, wherein the memory encodes one or more programs causing the one or more processor units to further perform the steps of
inputting measured amounts of a plurality of cellular constituents in a first cell of said cell type or of said organism exhibiting a phenotype that is a first profile; and
inputting measured amounts of a plurality of cellular constituents in a second cell of said cell type or of said organism having a genetic perturbation that is a landmark profile
before the step of determining the degree of similarity between said first profile and said landmark profile.

59. A system for determining if a genotype associated with a phenotype of interest is present in a cell type or organism, said system comprising:
(a) one or more memory units; and
(b) one or more processor units interconnected with the one or more memory units,
wherein the one or more memory units encodes one or more programs causing the one or more processor units to perform a method comprising
comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and
wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

60. The system of claim 59, wherein the phenotype is desirable.

61. The system of claim 59, wherein the phenotype is undesirable.

62. The system of claim 54, 57, or. 59, wherein said programs further cause the one or more processor units to perform a step of converting expression data into expression values of a plurality of sets of co-varying genes.

63. The system of claim 54, 57, or 59, wherein said programs further cause the one or more processor units to perform a step of predicting said predicted profile, and wherein said first profile is at a first developmental stage or first condition and said predicted profile is at a second, different developmental stage or condition more similar to the developmental stage or condition of said second cell than said first cell.

64. The system of claim 59, wherein the measured amounts of the plurality of cellular constituents in said first cell of said cell type or of said organism are determined in comparison to a wild-type cell of said cell type or of said organism, and wherein the measured amounts of the plurality of cellular constituents in said second cell of said cell type or of said organism are determined in comparison to a wild-type cell of said cell type or of said organism.

65. The method of claim 59, wherein the measured amounts of the plurality of cellular constituents in said first cell of said cell type or of said organism and the measured amounts the plurality of cellular constituents in said second cell of said cell type or of said organism are absolute amounts of the pluralities of the cellular constituents.

66. A computer program product for use in conjunction with a computer having one or more memory units and one or more processor units, the computer program product comprising a computer readable storage medium having a computer program mechanism encoded thereon, wherein said computer program mechanism may be loaded into the one or more memory units of a computer and cause the one or more processor units of the computer to execute the step of:
comparing a first profile or a predicted profile derived therefrom to a database comprising a plurality of landmark profiles to determine whether one or more landmark profiles known to be indicative of the presence or absence of a genotype associated with the phenotype of interest is similar to said first or predicted profile; wherein said first profile comprises measured amounts of a plurality of cellular constituents in a first cell of said cell type or of said organism; wherein each landmark profile comprises measured amounts of a plurality of cellular constituents in a second cell of said cell type or type of organism having a perturbation to a known gene or its encoded RNA or protein; and
wherein determining that the landmark profiles known to be indicative of the presence of said genotype are similar to said first or predicted profile, is indicative of the presence of said genotype associated with the phenotype of interest in the cell type or organism; and wherein determining that the landmark profiles known to be indicative of the absence of said genotype are similar to said first or predicted profile, is indicative of the absence of said genotype associated with the phenotype of interest in the cell type or organism.

67. The computer program product of claim 66, further comprising the step of converting expression data into expression values of a plurality of sets of co-varying genes.

68. The computer program product of claim 66, wherein the measured amounts of the plurality of cellular constituents in said first cell of said cell type or of said organism are determined in comparison to a wild-type cell of said cell type or of said organism, and wherein the measured amounts of the plurality of cellular constituents in said second cell of said cell type or of said organism are determined in comparison to a wild-type cell of said cell type or of said organism.

69. The computer program product of claim 66, wherein the measured amounts of the plurality of cellular constituents in said first cell of said cell type or of said organism and the measured amounts of the plurality of cellular constituents in said second cell of said cell type or of said organism are absolute amounts of the pluralities of cellular constituents.

70. The method of claim 1, 2, 3 or 4, wherein said one or more landmark profiles determined to be most similar to said first or predicted profile is a consensus profile associated with a perturbation to said known gene.

71. The method of claim 31 or 32, wherein said one or more landmark profiles known to be indicative of the presence or absence of a genotype associated with a phenotype of interest is a consensus profile associated with the presence or absence of said genotype associated with said phenotype of interest.
